SEQUENCE LISTING (105 sequences)

SEQ ID NO: 1 - protein, 869 aa, Homo sapiens
MRELVNIPLVHILTLVAFSGTEKLPKAPVITTPLETVDALVEEVATFMCAVESYPQPEISWTRNKILIKLFDTRYSIRENGQLLTILSVEDSDDGIYCCTANNGVGGAVESCGALQVKMKPKITRPPINVKIIEGLKAVLPCTTMGNPKPSVSWIKGDSPLRENSRIAVLESGSLRIHNVQKEDAGQYRCVAKNSLGTAYSKVVKLEVEVFARILRAPESHNVTFGSFVTLHCTATGIPVPTITWIENGNAVSSGSIQESVKDRVIDSRLQLFITKPGLYTCIATNKHGEKFSTAKAAATISIAEWSKPQKDNKGYCAQYRGEVCNAVLAKDALVFLNTSYADPEEAQELLVHTAWNELKVVSPVCRPAAEALLCNHIFQECSPGVVPTPIPICREYCLAVKELFCAKEWLVMEEKTHRGLYRSEMHLLSVPECSKLPSMHWDPTACARLPHLDYNKENLKTFPPMTSSKPSVDIPNLPSSSSSSFSVSPTYSMTVIISIMSSFAIFVLLTITTLYCCRRRKQWKNKKRESAAVTLTTLPSELLLDRLHPNPMYQRMPLLLNPKLLSLEYPRNNIEYVRDIGEGAFGRVFQARAPGLLPYEPFTMVAVKMLKEEASADMQADFQREAALMAEFDNPNIVKLLGVCAVGKPMCLLFEYMAYGDLNEFLRSMSPHTVCSLSHSDLSMRAQVSSPGPPPLSCAEQLCIARQVAAGMAYLSERKFVHRDLATRNCLVGENMVVKIADFGLSRNIYSADYYKANENDAIPIRWMPPESIFYNRYTTESDVWAYGVVLWEIFSYGLQPYYGMAHEEVIYYVRDGNILSCPENCPVELYNLMRLCWSKLPADRPSFTSIHRILERMCERAEGTVSV

SEQ ID NO: 2 - protein, 121 aa, synthetic construct
EVQLVESGGGLVKPGGSLRLSCAASGFTFSSYSMNWVRQAPGKGLEWVSSISSSSRYIYYADSVKGRFTISRDNANNSLYLQMNSLRAEDTAIYYCAGSGSSYYYYGVDVWGQGTTVTVSS

SEQ ID NO: 3 - protein, 8 aa, synthetic construct
GFTFSSYS

SEQ ID NO: 4 - protein, 8 aa, synthetic construct
ISSSSRYI

SEQ ID NO: 5 - protein, 14 aa, synthetic construct
AGSGSSYYYYGVDV

SEQ ID NO: 6 - protein, 108 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPLTFGGGTKVEIK

SEQ ID NO: 7 - protein, 7 aa, synthetic construct
QSVSSSY

SEQ ID NO: 8 - skipped

SEQ ID NO: 9 - protein, 9 aa, synthetic construct
QQYGSSPLT

SEQ ID NO: 10 - protein, 117 aa, synthetic construct
QVQLQESGPGLVKPSETLSLTCTVSGDSISSYYWSWIRQPAGKGLEWIGRIYTSGNTNYNPSLKSRVTMSVDTSKNQFSLKLSSVTAADTAVYYCTREGLLGAFDIWGQGTMVTVSS

SEQ ID NO: 11 - protein, 8 aa, synthetic construct
GDSISSYY

SEQ ID NO: 12 - protein, 7 aa, synthetic construct
IYTSGNT

SEQ ID NO: 13 - protein, 11 aa, synthetic construct
TREGLLGAFDI

SEQ ID NO: 14 - protein, 111 aa, synthetic construct
DIVMTQSPLSLPVTPGEPASISCRSSQSLLHSNGYNYLDWYLQKPGQSPQLLIYLGSYRASGVPDRFSGSGSGTDFTLKISRVEAEDVGVYYCMQALQTITFGQGTRLEIK

SEQ ID NO: 15 - protein, 11 aa, synthetic construct
QSLLHSNGYNY

SEQ ID NO: 16 - skipped

SEQ ID NO: 17 - protein, 8 aa, synthetic construct
MQALQTIT

SEQ ID NO: 18 - protein, 123 aa, synthetic construct
QVQLQESGPGLVKPSQTLSLTCTVSGGSISSGGYCWSWIRQHPGKGLEWIGYIYYSGSTYYNPSLKSRVTISEDTSKNQFSLKLSSVTAADTAVYYCARGGRWLQLRDVFDIWGQGTMVTVSS

SEQ ID NO: 19 - protein, 10 aa, synthetic construct
GGSISSGGYC

SEQ ID NO: 20 - protein, 7 aa, synthetic construct
IYYSGST

SEQ ID NO: 21 - protein, 7 aa, synthetic construct
IYYSGST

SEQ ID NO: 22 - protein, 108 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAAYHCQQHSSSPLTFGGGTKVEIK

SEQ ID NO: 23 - protein, 7 aa, synthetic construct
QSVSSSY

SEQ ID NO: 24 - skipped

SEQ ID NO: 25 - protein, 9 aa, synthetic construct
QQHSSSPLT

SEQ ID NO: 26 - protein, 118 aa, synthetic construct
QVHLVQSGAEVKKPGASVKVSCKASGYTFTGYYMHWVRQAPGQGLEWMGWINPYSGGTSYAQKFQGRVTMTRDTSISTAYMELSRLISDDTAVYYCASGLGISAFDIWGQGTMVTVSS

SEQ ID NO: 27 - protein, 8 aa, synthetic construct
GYTFTGYY

SEQ ID NO: 28 - protein, 8 aa, synthetic construct
INPYSGGT

SEQ ID NO: 29 - protein, 11 aa, synthetic construct
ASGLGISAFDI

SEQ ID NO: 30 - protein, 108 aa, synthetic construct
SYVLTQPPSVSVAPGQTARITCGGNNIGSKSVHWYQQKPGQAPVLVVYDDSDRPSGIPERFSGSNSGNTATLTISRVEAGDEADYYCQVWDRSSDHYVFGTGTKVTVL

SEQ ID NO: 31 - protein, 6 aa, synthetic construct
NIGSKS

SEQ ID NO: 32 - skipped

SEQ ID NO: 33 - protein, 11 aa, synthetic construct
QVWDRSSDHYV

SEQ ID NO: 34 - protein, 118 aa, synthetic construct
QVQLVQSGAEVKKPGASVKVSCKASGYTFTGYYMHWVRQAPGQGLEWMGWINPNSGGTNYAQKFQGRVTMTRDTSISTAYMELTRLRSDDTAVYYCASGLGISAFDIWGQGTMVTVSS

SEQ ID NO: 35 - protein, 8 aa, synthetic construct
GYTFTGYY

SEQ ID NO: 36 - protein, 8 aa, synthetic construct
INPNSGGT

SEQ ID NO: 37 - protein, 11 aa, synthetic construct
ASGLGISAFDI

SEQ ID NO: 38 - protein, 108 aa, synthetic construct; identical to SEQ ID NO: 30

SEQ ID NO: 39 - protein, 6 aa, synthetic construct
NIGSKS

SEQ ID NO: 40 - protein, 6 aa, synthetic construct
NIGSKS

SEQ ID NO: 41 - protein, 11 aa, synthetic construct
QVWDRSSDHYV

SEQ ID NO: 42 - protein, 118 aa, synthetic construct
QVQLVQSGAEVKKPGASVRVSCKASGYTFTGYYMHWVRQAPGQGLEWMGWINPNSGGTNYAQKFQGRVTMTRDTSISTAYMELSRLRSDDTAVYYCASGLGISAFDIWGQGTMVTVSS

SEQ ID NO: 43 - protein, 8 aa, synthetic construct
GYTFTGYY

SEQ ID NO: 44 - protein, 8 aa, synthetic construct
INPNSGGT

SEQ ID NO: 45 - protein, 11 aa, synthetic construct
ASGLGISAFDI

SEQ ID NO: 46 - protein, 108 aa, synthetic construct
SYVLTQPPSVSVAPGQTARITCGGNNIGSKSVHWYQQKPGQAPVLVVYDDSDRPSGIPERFSGSNSGNTATLTISRVEAGDEADYYCQVWDSSSDHYVFGTGTKVTVL

SEQ ID NO: 47 - protein, 6 aa, synthetic construct
NIGSKS

SEQ ID NO: 48 - skipped

SEQ ID NO: 49 - protein, 11 aa, synthetic construct
QVWDSSSDHYV

SEQ ID NO: 50 - protein, 118 aa, synthetic construct; identical to SEQ ID NO: 42

SEQ ID NO: 51 - protein, 8 aa, synthetic construct
GYTFTGYY

SEQ ID NO: 52 - protein, 8 aa, synthetic construct
INPNSGGT

SEQ ID NO: 53 - protein, 11 aa, synthetic construct
ASGLGISAFDI

SEQ ID NO: 54 - protein, 107 aa, synthetic construct
DIQMTQSPSTLSASVGDRVTITCRASQSIDNWLAWYQQKPGKAPKLLIYKASSLESGVPSRFSGSGSGTEFILTISSLQPDDFATYYCQQYNSHSWTFGQGTKVEIK

SEQ ID NO: 55 - protein, 6 aa, synthetic construct
QSIDNW

SEQ ID NO: 56 - skipped

SEQ ID NO: 57 - protein, 9 aa, synthetic construct
QQYNSHSWT

SEQ ID NO: 58 - protein, 118 aa, synthetic construct
QVQLVQSGAEVKKPGASVKVSCKASGYTFTGYYMHWVRQAPGQGLEWMGWINPHSGGTNYAQKFQGRVTMTRDTSISTAYMELSRLRSDDTAVYSCASGLGISAFDIWGQGTMVTVSS

SEQ ID NO: 59 - protein, 8 aa, synthetic construct
GYTFTGYY

SEQ ID NO: 60 - protein, 8 aa, synthetic construct
INPHSGGT

SEQ ID NO: 61 - protein, 11 aa, synthetic construct
ASGLGISAFDI

SEQ ID NO: 62 - protein, 108 aa, synthetic construct; identical to SEQ ID NO: 46

SEQ ID NO: 63 - protein, 6 aa, synthetic construct
NIGSKS

SEQ ID NO: 64 - skipped

SEQ ID NO: 65 - protein, 11 aa, synthetic construct
QVWDSSSDHYV

SEQ ID NO: 66 - protein, 116 aa, synthetic construct
EVQLVESGGGLVKPGGSLRLSCAASGFTFSSYSMNWVRQAPGKGLEWVSSIGSSSGYIFYADSVKGRFTISRDNAKNSLYLQMNSLRAEDTAVYFCARDGGNFDYWGQGTLVTVSS

SEQ ID NO: 67 - protein, 8 aa, synthetic construct
GFTFSSYS

SEQ ID NO: 68 - protein, 8 aa, synthetic construct
IGSSSGYI

SEQ ID NO: 69 - protein, 9 aa, synthetic construct
ARDGGNFDY

SEQ ID NO: 70 - protein, 118 aa, synthetic construct
QPVLTQPSSHSASSGASVRLTCMLSSGFSVGDFWIRWYQQKPGNPPRYLLYYHSDSNKGQGSGVPSRFSGSNDASANAGILRISGLQPEDEADYYCGSWHTDSKTHWVFGGGTKLTVL

SEQ ID NO: 71 - protein, 9 aa, synthetic construct
SGFSVGDFW

SEQ ID NO: 72 - protein, 7 aa, synthetic construct
YHSDSNK

SEQ ID NO: 73 - protein, 12 aa, synthetic construct
GSWHTDSKTHWV

SEQ ID NO: 74 - protein, 119 aa, synthetic construct
EVQLLESGGGLVQPGGSLRLSCAASGFTFNSSAMSWVRQAPGKGLEWVSVISGSGGSTYYADSVKGHFTISRDNSKNTLYLQMNSLRAEDTAVYYCAKKELGDWYFDLWGRGTLVTVSS

SEQ ID NO: 75 - protein, 8 aa, synthetic construct
GFTFNSSA

SEQ ID NO: 76 - protein, 8 aa, synthetic construct
ISGSGGST

SEQ ID NO: 77 - protein, 12 aa, synthetic construct
AKKELGDWYFDL

SEQ ID NO: 78 - protein, 113 aa, synthetic construct
DIVMTQSPDSLAVSLGERATINCKSSQSVLYSSNNKNYLAWYQQRPGQPPKLLIYWASTRESGVPDRFSGSGSGTDFTLTISSLQAEDVAIYYCQQYYSIPPTFGQGTKVEIK

SEQ ID NO: 79 - protein, 12 aa, synthetic construct
QSVLYSSNNKNY

SEQ ID NO: 80 - skipped

SEQ ID NO: 81 - protein, 9 aa, synthetic construct
QQYYSIPPT

SEQ ID NO: 82 - protein, 124 aa, synthetic construct
EVQLVESGGGLVKPGGSLRLSCAASGFTFSSYDMNWVRQAPGKGLEWVSSISSSSSYIYYADSVKGRFTISRDNAKNSLYLQMNSLRAEDTAVYYCARDSYDSSGYYLRPFDYWGQGTLVTVSS

SEQ ID NO: 83 - protein, 8 aa, synthetic construct
GFTFSSYD

SEQ ID NO: 84 - protein, 8 aa, synthetic construct
ISSSSSYI

SEQ ID NO: 85 - protein, 17 aa, synthetic construct
ARDSYDSSGYYLRPFDY

SEQ ID NO: 86 - protein, 107 aa, synthetic construct; identical to SEQ ID NO: 54

SEQ ID NO: 87 - protein, 6 aa, synthetic construct
QSIDNW

SEQ ID NO: 88 - skipped

SEQ ID NO: 89 - protein, 9 aa, synthetic construct
QQYNSHSWT

SEQ ID NO: 90 - protein, 118 aa, synthetic construct
EVQLVESGGGLVQPGGSLRLSCAASGFTFSSYWMHWVRQAPGKGLVWVSRINSDGSSTSYADSVKGRFTISRDNAKNTLYLQMNSLRAEDTAVYYCARGGSGSYFDYWGQGTLVTVSS

SEQ ID NO: 91 - protein, 8 aa, synthetic construct
GFTFSSYW

SEQ ID NO: 92 - protein, 8 aa, synthetic construct
INSDGSST

SEQ ID NO: 93 - protein, 11 aa, synthetic construct
ARGGSGSYFDY

SEQ ID NO: 94 - protein, 112 aa, synthetic construct
DVVMTQSPLSLPVTLGQPASVSCRSSQSLVYSDGNTYLNWFQQRPGQSPRRLIYKVSNRDSGVPDRFSGSGSGTDFTLKISRVEAEDVGVYYCMQGTHWPLTFGGGTKVEIK

SEQ ID NO: 95 - protein, 11 aa, synthetic construct
QSLVYSDGNTY

SEQ ID NO: 96 - skipped

SEQ ID NO: 97 - protein, 9 aa, synthetic construct
MQGTHWPLT

SEQ ID NO: 98 - protein, 118 aa, synthetic construct; identical to SEQ ID NO: 90

SEQ ID NO: 99 - protein, 8 aa, synthetic construct
GFTFSSYW

SEQ ID NO: 100 - protein, 8 aa, synthetic construct
INSDGSST

SEQ ID NO: 101 - protein, 11 aa, synthetic construct
ARGGSGSYFDY

SEQ ID NO: 102 - protein, 110 aa, synthetic construct
QSVLTQPPSVSGAPGQRVTISCTGSSSNIGAGFDVHWYQQLPGTAPKLLIYFTNNRPSGVPDRFSGSKSGTSASLAITGLQAEDESDYYCQSYDSSLRWVFGGGTKLTVL

SEQ ID NO: 103 - protein, 9 aa, synthetic construct
SSNIGAGFD

SEQ ID NO: 104 - skipped

SEQ ID NO: 105 - protein, 10 aa, synthetic construct
QSYDSSLRWV

(19) United States
(12) Patent Application Publication
(10) Pub. No.: US 2026/0191984 A1
(43) Pub. Date: Jul. 9, 2026

(54) Anti-MuSK Antibody Conjugates

(71) Applicant: MABWELL THERAPEUTICS, INC., San Diego, CA (US)

(72) Inventors: Yubin Wang, San Diego, CA (US); Xin Du, La Jolla, CA (US); Lei Huang, San Diego, CA (US)

(21) Appl. No.: 19/441,160

(22) Filed: Jan. 6, 2026

Related U.S. Application Data
(60) Provisional application No. 63/742,239, filed on Jan. 6, 2025.

(51) Int. Cl. A61K 47/68; C07K 16/28

(52) U.S. Cl. CPC A61K 47/6849; A61K 47/6811; A61K 47/6889; C07K 16/2863; C07K 2317/40

(57) ABSTRACT

The present disclosure relates to antibody peptide conjugates with a means to bind MuSK, such as an anti-MuSK antibody, and a PRAD peptide. The present disclosure also relates to an antibody-drug conjugate including an anti-Musk antibody and a drug, and optionally a PRAD peptide.

### CROSS-REFERENCE

This application claims priority to U.S. Provisional Patent Application No. 63/742,239, filed on Jan. 6, 2025, the contents of which are incorporated herein by reference in their entirety.

### SEQUENCE LISTING

The computer-readable Sequence Listing submitted on Jan. 6, 2026, and identified as follows: 88,150 bytes ST.26 XML document file name “Sequence_Listing.xml,” created on Jan. 5, 2026, is incorporated herein by reference in its entirety.

### BACKGROUND

The present disclosure relates generally to antibody peptide conjugates including an antibody that binds to Muscle-Specific Kinase (MuSK) and a peptide. The disclosure further relates to an antibody-drug conjugate (ADC) comprising an anti-MuSK antibody, a drug, and, optionally a peptide.

MuSK is a receptor tyrosine kinase crucial for the function of neuromuscular junction (NMJ). Hubbard et al., Biochimica et Biophysica Acta (BBA)-Proteins and Proteomics, 1834(10): 2166-2169 (2013). MuSK is essential during early stages of NMJ development, mediating the clustering of acetylcholine receptors (AChRs) on NMJ, which is essential for the formation and maintenance of functional NMJ. DeChiara et al., Cell, 85: 501-512 (1996); Glass et al., Cell, 85: 513-523 (1996); Flanagan-Steet et al., Development, 132: 4471-4481 (2005); Panzer et al., J. Neurosci., 26: 934-947 (2006).

MuSK is a single-pass transmembrane glycoprotein of ~120 kDa with an extracellular region comprising three Ig-like domains (Ig1, Ig2, Ig3) and one frizzled-like cysteine-rich domain (Fz-CRD). A single transmembrane helix connects the extracellular domains to the intracellular tyrosine kinase domain. Its intracellular region contains several tyrosine phosphorylation sites which are crucial for MuSK activation. Hubbard et al., Biochimica et Biophysica Acta, 1834: 2166-2169 (2013).

MuSK is expressed on the membrane of muscle cells. Zhu et al., J. Neurosci., 28(7): 1688-1696 (2008). Activation of MuSK by agrin, a neuronally derived heparin sulfate proteoglycan, and lipoprotein-related protein 4 (LRP4), the agrin receptor, leads to clustering of acetylcholine receptors (AChRs) on the postsynaptic side of the NMJ, enabling neuromuscular transmission and muscle contraction.

At the neuromuscular junction (NMJ), collagen Q (CoIQ) and acetylcholinesterase (AChE) play critical roles in maintaining efficient synaptic transmission and ensuring proper muscle function. CoIQ is a specialized collagen that anchors AChE to the synaptic basal lamina in the NMJ, facilitating its localization in the extracellular matrix. CoIQ forms a triple-helical structure with specific binding sites that allow it to attach to tetramers of AChE, creating a stable complex called CoIQ-tailed AChE or asymmetric AChE, the predominant AChE species at the NMJ. Additionally, CoIQ's C-terminal region interacts with extracellular matrix components such as MuSK complex and perlecan, anchoring the CoIQ-AChE complex at the NMJ. This anchoring enables AChE to remain in proximity to the synaptic cleft. This anchoring is essential for AChE to perform its primary function of rapidly degrading acetylcholine (ACh) after it is released into the synaptic cleft and activates acetylcholine receptor (AChR), thus preventing continuous muscle contraction and ensuring precise timing of muscle relaxation. Additionally, CoIQ stabilizes the NMJ structure by binding to components of the extracellular matrix, contributing to the organization of the synaptic environment. Together, the interaction between CoIQ and AChE enables finely tuned motor responses and controlled muscle movement, with disruptions in either protein potentially leading to neuromuscular disorders characterized by impaired muscle strength and control.

A need exists for therapies that overcome these challenges and restore NMJ function. For example, a need exists to deliver therapeutics specifically to the NMJ.

### BRIEF DESCRIPTION OF THE FIGURES

FIG. 1A depicts Western Blot analysis of MuSK phosphorylation in C2C12 myotubes treated with anti-MuSK antibodies; FIG. 1B depicts quantification of the ratio of phospho-MuSK to total MuSK (N=3).

FIG. 2A depicts Western Blot analysis of MuSK phosphorylation in C2C12 myotubes treated with Agrin in the presence or absence of anti-MuSK antibodies; FIG. 2B depicts quantification of the ratio of phospho-MuSK to total MuSK (N=2).

FIG. 3 shows a graph of an ELISA assay of mu07D09A, mu07D09A-PRADₛₕₒᵣₜ and mu07D09A-PRAD_{long} against huMuSK ECD.

FIG. 4 shows a graph of an AChE binding assay depicting AChE activity on a plate coated with huMuSK ECD and treated with mixture of AChE and anti-MuSK antibody with or without PRAD peptides.

FIG. 5 shows fluorescent IHC staining of paraffin-embedded human skeletal muscle sections after treatment with the present antibody peptide conjugate.

FIG. 6 shows fluorescent IHC staining of paraffin-embedded human skeletal muscle sections after treatment with the present antibody peptide conjugate and AChE.

FIG. 7 shows a graph of ELISA assay of hu02C13A, hu07D09A, hu02C13A-PRAD and hu07D09A-PRAD against huMuSK ECD.

FIG. 8 shows a graph of AChE binding assay depicting AChE activity on a plate coated with huMuSK ECD and treated with mixture of AChE and anti-MuSK antibody with or without PRAD peptides.

FIGS. 9A to 9D show panels of quantitative analyses of IHC staining of mouse skeletal muscle sections with Bungarotoxin Alexa Fluor™ 647, anti-acetylcholinesterase antibody, and goat anti-human IgG conjugated Alexa Fluor™ 488. FIG. 9A depicts the percentage of human IgG positive NMJs. FIG. 9B depicts the percentage of huIgG and AChE positive NMJs. FIG. 9C depicts the percentage of AChE positive NMJs. FIG. 9D depicts AChE intensity at NMJ. N=3. * p<0.05 hu02C13A-PRAD vs. Vehicle. Unpaired t-test.

FIGS. 10A to 10D depict quantitative analysis of NMJ morphology on mouse skeletal muscle sections(n=3). FIG. 10A shows the total NMJ numbers per image; FIG. 10B shows the average NMJ area; FIG. 10C depicts the average NMJ circularity; and FIG. 10D depicts the average NMJ ellipticity.

FIG. 11A and FIG. 11B depict pharmacokinetics (PK) of hu02C13A-PRAD and hu02C13A-PRAD (Cys) in C57BL/6J mice. FIG. 11A shows the time course of the drug concentration in serum. FIG. 11B depicts the PK profiles of hu02C13A-PRAD and hu02C13A-PRAD (Cys) in serum.

FIGS. 12A to 12D show Western blots from skeletal muscle homogenates seven days post-administration of hu02C13A-PRAD. FIG. 12A shows MuSK and AChE levels in normal and denervated muscle homogenate at 7 days after hu02C13A-PRAD injection. FIG. 12B depicts pull-down of hu02C13-PRAD and AChE from muscle homogenate collected at 7 days after hu02C13A-PRAD injection. FIG. 12C shows MuSK and AChE levels in normal (N) and denervated (D) muscle homogenate at 7 days after hu02C13A-PRAD (Cys) injection. FIG. 12D depicts pull-down of hu02C13-PRAD (Cys) and AChE from muscle homogenate collected at 7 days after hu02C13A-PRAD (Cys) injection.

### DETAILED DESCRIPTION

This description is not provided to limit the disclosure to the embodiments described herein, but rather to explain and teach various principles to enable one of ordinary skill in the art to understand these principles and, with that understanding, be able to apply them to practice not only the embodiments described herein, but also other embodiments that may come to mind in accordance with these principles. The scope of the instant disclosure is intended to cover all such embodiments that may fall within the scope of the appended claims, either literally or under the doctrine of equivalents. All references cited in the present disclosure are incorporated by reference in their entirety.

### Definitions

The following terms or definitions are provided solely to aid in the understanding of the present disclosure. Unless specifically defined herein, all terms used herein have the same meaning as would be understood to one of ordinary skill in the art.

Unless indicated otherwise, all methods, steps, and techniques that are not specifically described in detail can be performed and have been performed in a manner known to a skilled artisan and described in the prior art, such as Sambrook et al., Molecular Cloning: A Laboratory Manual 2^{nd} ed., Cold Spring Harbor Press, Plainsview, NY (1989) and Ausubel et al., Current Protocols in Molecular Biology (Supplement 47), John Wiley & Sons, NY (1999).

As used herein, the singular forms “a”, “an”, and “the” include both singular and plural unless the context clearly indicates otherwise.

“Antibody” refers in the broadest sense to a polypeptide or combination of polypeptides that recognizes and binds to an antigen through one or more immunoglobulin variable regions, where the immunoglobulin variable regions may be naturally occurring or non-naturally occurring, e.g., as a result of engineering, chimerization, humanization, optimization, CDR-grafting, or affinity maturation.

An “antibody” as disclosed herein can be a whole (intact, full length) antibody, a single chain antibody, or an antigen binding fragment with one or two chains. An antibody comprises at least sufficient complementarity determining regions (CDR), interspersed with framework regions (FR), for the antibody to recognize and bind to an antigen. An anti-MuSK antibody disclosed herein may be, but is not limited to, at least one of a monoclonal antibody, a polyclonal antibody, a humanized antibody, a fully human antibody, a chimeric antibody, a single chain antibody, a Fab fragment, a single-chain variable fragment (scFv), an aptamer, a single-domain antibody (VHH or nanobody), a recombinant antibody, a modified antibody having peptide/other moieties attached to antibody and/or additional amino acids added the N- or C-terminus, or other MuSK fragment or variant. Whole antibody, full length antibody, intact antibody, or equivalent terms are understood to refer to a polypeptide, in particular a glycoprotein, comprising at least two heavy chains (HCs) and two light chains (LCs) interconnected by disulfide bonds. Each HC is comprised of a heavy chain variable region (VH) and an HC constant region (CH), and each light chain is comprised of a light chain variable region (VL) and an LC constant region (CL). The HC and LC variable regions, VH and VL, include a binding domain that interacts with an antigen. The VH and VL regions can be further subdivided into CDR regions characterized by hypervariability, interspersed with FR regions that are typically more conserved. Each VH and VL is typically composed of three CDRs and four FRs arranged from amino-terminus to carboxy-terminus in the following order: FR1, CDR1, FR2, CDR2, FR3, CDR3, FR4. The constant regions of the antibodies may mediate the binding of the immunoglobulin to host tissues or factors, including various cells of the immune system and the classical complement system.

As used herein, the term “sequence identity” is defined as a relationship between two or more amino acid (polypeptide or protein) sequences or two or more nucleic acid (polynucleotide) sequences, as determined by comparing the sequences. The identity between two amino acid sequences is preferably defined by assessing their identity within a whole SEQ ID NO as identified herein. By way of example, if a first sequence is identical to a second sequence, they have 100% sequence identity; and if a first sequence contains 100 residues and a second sequence has 100 residues, but differs in only on residue, they have 99% sequence identity. Software tools such as NCBI Blast (Basic Local Alignment Search Tool) and others provide the sequence identity as referred to in the present disclosure. Assessment of sequence identity may also be conducted on part of a provided sequence. For example, in embodiments, “part of a provided sequence” may mean at least 50% of the length of the SEQ ID NO, or at least 60%, or at least 70%, or at least 80%, or at least 90% after the respective sequence alignment is performed. Unless specified, “sequence identity” is being referred to in the context of the entire SEQ ID NO.

As used herein, a “conservative amino acid substitution” is an amino acid substitution in which an amino acid residue is replaced with another amino acid residue of similar chemical structure (e.g., hydrophilicity, degree and distribution of charged regions). Such substitutions are recognized in the art as typically involving a minor change with no or essentially no influence on the function, activity or other biological properties of the polypeptide. These minor changes can be identified, in part, by considering the hydropathic index of amino acids, as understood in the art. Kyte et al., J. Mol. Biol. 157:105-132 (1982). The hydropathic index of an amino acid is based on a consideration of its hydrophobicity and charge. In preferred embodiments, a conserved amino acid substitution involves substitution of amino acids having hydropathic indexes of ±2.

Within the context of this disclosure, each amino acid sequence described herein can also be interpreted as an amino acid sequence with a sequence identity of at least 80%, 81%, 82%, 83%, 84%, 85%, 86%, 87%, 88%, 89%, 90%, 91%, 92%, 93%, 94%, 95%, 96%, 97%, 98%, 99% or more identity with the given amino acid sequence. In a preferred embodiment, sequence identity is determined by comparing the whole length of the sequences as identified herein.

Within the context of this disclosure, each amino acid sequence described herein can also be interpreted as containing one conservative substitution of a CDR sequence; or alternatively, one, two, three, four, five, six, or seven conservative amino acid sequence substitutions in non-CDR sequences.

“Variant” is used herein to describe a peptide or polypeptide that differs in amino acid sequence by the insertion, deletion, or conservative substitution of amino acids, but retain at least one biological activity. Representative examples of “biological activity” include the ability to be bound by a specific antibody. Variant is also used herein to describe a protein with an amino acid sequence that is substantially identical to a referenced protein with an amino acid sequence that retains at least one biological activity. “Variant” also can be used to refer to an antigenically reactive fragment of an anti-MuSK antibody that differs from the corresponding fragment of anti-MuSK antibody in amino acid sequence but is still antigenically reactive and can compete with the corresponding fragment of anti-MuSK antibody for binding with MuSK. “Variant” also can be used to describe a polypeptide or a fragment thereof that has been differentially processed, such as by proteolysis, phosphorylation, or other post-translational modification, yet retains its antigen reactivity.

The term “vector,” as used herein, refers to a nucleic acid molecule capable of propagating a nucleic acid to which the vector sequence is linked, in a host cell in which the vector is introduced.

MuSK may have the following amino acid sequence: MRELVNIPLV HILTLVAFSG TEKLPKAPVI TTPLETVDAL VEEVATFMCA VESYPQPEIS WTRNKILIKL FDTRYSIREN GQLLTILSVE DSDDGIYCCT ANNGVGGAVE SCGALQVKMK PKITRPPINV KIIEGLKAVL PCTTMGNPKP SVSWIKGDSP LRENSRIAVL ESGSLRIHNV QKEDAGQYRC VAKNSLGTAY SKVVKLEVEV FARILRAPES HNVTFGSFVT LHCTATGIPV PTITWIENGN AVSSGSIQES VKDRVIDSRL QLFITKPGLY TCIATNKHGE KFSTAKAAAT ISIAEWSKPQ KDNKGYCAQY RGEVCNAVLA KDALVFLNTS YADPEEAQEL LVHTAWNELK VVSPVCRPAA EALLCNHIFQ ECSPGVVPTP IPICREYCLA VKELFCAKEW LVMEEKTHRG LYRSEMHLLS VPECSKLPSM HWDPTACARL PHLDYNKENL KTFPPMTSSK PSVDIPNLPS SSSSSFSVSP TYSMTVIISI MSSFAIFVLL TITTLYCCRR RKQWKNKKRE SAAVTLTTLP SELLLDRLHP NPMYQRMPLL LNPKLLSLEY PRNNIEYVRD IGEGAFGRVF QARAPGLLPY EPFTMVAVKM LKEEASADMQ ADFQREAALM AEFDNPNIVK LLGVCAVGKP MCLLFEYMAY GDLNEFLRSM SPHTVCSLSH SDLSMRAQVS SPGPPPLSCA EQLCIARQVA AGMAYLSERK FVHRDLATRN CLVGENMVVK IADFGLSRNI YSADYYKANE NDAIPIRWMP PESIFYNRYT TESDVWAYGV VLWEIFSYGL QPYYGMAHEE VIYYVRDGNI LSCPENCPVE LYNLMRLCWS KLPADRPSFT SIHRILERMC ERAEGTVSV (SEQ ID NO:1). MuSK may be a variant or fragment of SEQ ID NO:1. Exemplary variants include D38E, P344R, 1575T, M6051, A727V, V790M and M835V. Alternate variants include deletion, insertion, or substitution of the amino acid at position 27, 38, 100, 107, 159, 222, 344, 413, 575, 584, 605, 609, 629, 644, 664, 696, 727, 743, 782, 790, 819, 829, 835, or 858 of SEQ ID NO:1. As used herein, amino acids at positions 28-116 of SEQ ID NO:1 is the “Ig-1 domain”; amino acids at positions 121-205 of SEQ ID NO:1 is the “Ig-2 domain”; amino acids at positions 212-302 of SEQ ID NO:1 is the “Ig-3 domain”; amino acids at positions 312-450 of SEQ ID NO:1 is the “frizzled-like cysteine-rich domain (FZ) domain”; and amino acids at positions 575-856 of SEQ ID NO:1 is the “Protein Kinase domain.”

In a first aspect, the present disclosure describes an anti-MuSK antibody that is (i) capable of binding MuSK (SEQ ID NO:1) or a variant thereof, and (ii) provides a negligible effect on MuSK function. The anti-MuSK antibody may serve as an anchor in the NMJ, particularly without activating or inhibiting MuSK function as those terms are classically understood in the art. For example, agonistic anti-MuSK antibodies are known to increase function by sixty (60) fold. The antibodies of the present disclosure may provide some activation or inhibition of MuSK; however, such activation or inhibition is negligible, wherein the function of MuSK post binding is not increased or decreased by more than 50%, preferably 25%, more preferably 15%, and most preferably 10%. Alternatively, the present disclosure describes a means for binding MuSK that provides a negligible effect on MuSK function, wherein the function of MuSK post binding is not increased or decreased by more than 50%, preferably 25%, more preferably 15%, and most preferably 10%. In embodiments, the anti-MuSK antibody or binding means binds to the Fz domain of human MuSK. In other embodiments, the anti-MuSK antibody or binding means binds to the Ig-3 domain of human MuSK.

### The Anti-MuSK Antibody

An anti-MuSK antibody of the present disclosure may comprise a variable heavy chain comprising a CDR1 with the amino acid sequence set forth in SEQ ID NOs: 3, 11, 19, 27, 35, 43, 51, 59, 67, 75, 83, or 91, a CDR2 with the amino acid sequence set forth in SEQ ID NOs: 4, 12, 20, 28, 36, 44, 52, 60, 68, 76, 84, or 92; and a CDR3 with the amino acid sequence set forth in SEQ ID NOs: 5, 13, 21, 29, 37, 45, 53, 61, 69, 77, 85, or 93.

An anti-MuSK antibody of the present disclosure may comprise a variable heavy chain comprising the amino acid sequence set forth in SEQ ID NOs: 2, 10, 18, 26, 34, 42, 50, 58, 66, 74, 82, 90, or 98.

An anti-MuSK antibody of the present disclosure may comprise a variable light chain comprising a CDR1 with the amino acid sequence set forth in SEQ ID NOs: 7, 15, 23, 31, 39, 47, 55, 63, 71, 79, 87, or 95; a CDR2 with the amino acid sequence set forth in SEQ ID NOs: 8, 16, 24, 32, 40, 48, 56, 64, 72, 80, 88, or 96; and a CDR3 with the amino acid sequence set forth in SEQ ID NOs: 9, 17, 25, 33, 41, 49, 57, 65, 73, 81, 89, or 97.

An anti-MuSK antibody of the present disclosure may comprise a variable light chain comprising the amino acid sequence set forth in SEQ ID NOs: 6, 14, 22, 30, 38, 46, 54, 62, 70, 78, 86, or 94.

Exemplary anti-MuSK antibodies include 02C13 as defined in Table 1, 02012 as defined in Table 2, 05D18 as defined in Table 3, 06B20 as defined in Table 4, 06E17 as defined in Table 5, 07A13 (lambda) as defined in Table 6, 07A13 (lambda) as defined in Table 7, 07D09 as defined in Table 8, 09L18 as defined in Table 9, 11A04 as defined in Table 10, 11G12 as defined in Table 11, 12K18 (kappa) as defined in Table 12, 12K18 (lambda) as defined in Table 13.

TABLE 1

| SEQ | | |
|---|---|---|
| ID | | |
| NO: | | 02C13 |
| 2 | VH | EVQLVESGGGLVKPGGSLRLSCAASGFTFS |
| | | SYSMNWVRQAPGKGLEWVSSISSSSRYIYY |
| | | ADSVKGRFTISRDNANNSLYLQMNSLRAED |
| | | TAIYYCAGSGSSYYYYGVDVWGQG |
| | | TTVTVSS |
| 3 | HC | GFTFSSYS |
| | CDR1 | |
| 4 | HC | ISSSSRYI |
| | CDR2 | |
| 5 | HC | AGSGSSYYYYGVDV |
| | CDR3 | |
| 6 | VL | EIVLTQSPGTLSLSPGERATLSCRASQSVS |
| | | SSYLAWYQQKPGQAPRLLIYGASSRATGIP |
| | | DRFSGSGSGTDFTLTISRLEPEDFAVYYCQ |
| | | QYGSSPLTFGGGTKVEIK |
| 7 | LC | QSVSSSY |
| | CDR1 | |
| 8 | LC | GAS |
| | CDR2 | |
| 9 | LC | QQYGSSPLT |
| | CDR3 | |

TABLE 2

| SEQ | | |
|---|---|---|
| ID | | |
| NO: | | 02O12 |
| 10 | VH | QVQLQESGPGLVKPSETLSLTCTVSGDSISSYYWSWIRQ |
| | | PAGKGLEWIGRIYTSGNTNYNPSLKSRVTMSVDTSKNQ |
| | | FSLKLSSVTAADTAVYYCTREGLLGAFDIWGQGTMVT |
| | | VSS |
| 11 | HC | GDSISSYY |
| | CDR1 | |
| 12 | HC | IYTSGNT |
| | CDR2 | |
| 13 | HC | TREGLLGAFDI |
| | CDR3 | |
| 14 | VL | DIVMTQSPLSLPVTPGEPASISCRSSQSLLHSNGYNYLD |
| | | WYLQKPGQSPQLLIYLGSYRASGVPDRFSGSGSGTDFTL |
| | | KISRVEAEDVGVYYCMQALQTITFGQGTRLEIK |
| 15 | LC | QSLLHSNGYNY |
| | CDR1 | |
| 16 | LC | LGS |
| | CDR2 | |
| 17 | LC | MQALQTIT |
| | CDR3 | |

TABLE 3

| SEQ | | |
|---|---|---|
| ID | | |
| NO: | | 05D18 |
| 18 | VH | QVQLQESGPGLVKPSQTLSLTCTVSGGSISSGGYCWSWI |
| | | RQHPGKGLEWIGYIYYSGSTYYNPSLKSRVTISEDTSKN |
| | | QFSLKLSSVTAADTAVYYCARGGRWLQLRDVFDIWGQGT |
| | | MVTVSS |
| 19 | HC | GGSISSGGYC |
| | CDR1 | |
| 20 | HC | IYYSGST |
| | CDR2 | |
| 21 | HC | ARGGRWLQLRDVFDI |
| | CDR3 | |
| 22 | VL | EIVLTQSPGTLSLSPGERATLSCRASQSVSSSYLAWYQQ |
| | | KPGQAPRLLIYGASSRATGIPDRFSGSGSGTDFTLTISR |
| | | LEPEDFAAYHCQQHSSSPLTFGGGTKVEIK |
| 23 | LC | QSVSSSY |
| | CDR1 | |
| 24 | LC | GAS |
| | CDR2 | |
| 25 | LC | QQHSSSPLT |
| | CDR3 | |

TABLE 4

| SEQ | | |
|---|---|---|
| ID | | |
| NO: | | 06B20 |
| 26 | VH | QVHLVQSGAEVKKPGASVKVSCKASGYTFTGYYMHWV |
| | | RQAPGQGLEWMGWINPYSGGTSYAQKFQGRVTMTRDT |
| | | SISTAYMELSRLISDDTAVYYCASGLGISAFDIWGQG |
| | | TMVTVSS |
| 27 | HC | GYTFTGYY |
| | CDR1 | |
| 28 | HC | INPYSGGT |
| | CDR2 | |
| 29 | HC | ASGLGISAFDI |
| | CDR3 | |
| 30 | VL | SYVLTQPPSVSVAPGQTARITCGGNNIGSKSVHWYQQ |
| | | KPGQAPVLVVYDDSDRPSGIPERFSGSNSGNTATLTI |
| | | SRVEAGDEADYYCQVWDRSSDHYVFGTGTKVTVL |
| 31 | LC | NIGSKS |
| | CDR1 | |
| 32 | LC | DDS |
| | CDR2 | |
| 33 | LC | QVWDRSSDHYV |
| | CDR3 | |

TABLE 5

| SEQ | | |
|---|---|---|
| ID | | |
| NO: | | 06E17 |
| 34 | VH | QVQLVQSGAEVKKPGASVKVSCKASGYTFTGYYMHWV |
| | | RQAPGQGLEWMGWINPNSGGTNYAQKFQGRVTMTRDT |
| | | ISSTAYMELTRLRSDDTAVYYCASGLGISAFDIWGQG |
| | | TMVTVSS |
| 35 | HC | GYTFTGYY |
| | CDR1 | |
| 36 | HC | INPNSGGT |
| | CDR2 | |
| 37 | HC | ASGLGISAFDI |
| | CDR3 | |
| 38 | VL | SYVLTQPPSVSVAPGQTARITCGGNNIGSKSVHWYQQ |
| | | KPGQAPVLVVYDDSDRPSGIPERFSGSNSGNTATLTI |
| | | SRVEAGDEADYYCQVWDRSSDHYVFGTGTKVTVL |
| 39 | LC | NIGSKS |
| | CDR1 | |
| 40 | LC | NIGSKS |
| | CDR2 | |
| 41 | LC | QVWDRSSDHYV |
| | CDR3 | |

TABLE 6

| SEQ | | |
|---|---|---|
| ID | | |
| NO: | | 07A13 (lambda) |
| 42 | VH | QVQLVQSGAEVKKPGASVRVSCKASGYTFTGYYMHWV |
| | | RQAPGQGLEWMGWINPNSGGTNYAQKFQGRVTMTRDT |
| | | SISTAYMELSRLRSDDTAVYYCASGLGISAFDIWGQG |
| | | TMVTVSS |
| 43 | HC | GYTFTGYY |
| | CDR1 | |
| 44 | HC | INPNSGGT |
| | CDR2 | |
| 45 | HC | ASGLGISAFDI |
| | CDR3 | |
| 46 | VL | SYVLTQPPSVSVAPGQTARITCGGNNIGSKSVHWYQQ |
| | | KPGQAPVLVVYDDSDRPSGIPERFSGSNSGNTATLTI |
| | | SRVEAGDEADYYCQVWDSSSDHYVFGTGTKVTVL |
| 47 | LC | NIGSKS |
| | CDR1 | |
| 48 | LC | DDS |
| | CDR2 | |
| 49 | LC | QVWDSSSDHYV |
| | CDR3 | |

TABLE 7

| SEQ | | |
|---|---|---|
| ID | | |
| NO: | | 07A13 (kappa) |
| 50 | VH | QVQLVQSGAEVKKPGASVRVSCKASGYTFTGYYMHWV |
| | | RQAPGQGLEWMGWINPNSGGTNYAQKFQGRVTMTRDT |
| | | SISTAYMELSRLRSDDTAVYYCASGLGISAFDIWGQG |
| | | TMVTVSS |
| 51 | HC | GYTFTGYY |
| | CDR1 | |
| 52 | HC | INPNSGGT |
| | CDR2 | |
| 53 | HC | ASGLGISAFDI |
| | CDR3 | |
| 54 | VL | DIQMTQSPSTLSASVGDRVTITCRASQSIDNWLAWYQ |
| | | QKPGKAPKLLIYKASSLESGVPSRFSGSGSGTEFILT |
| | | ISSLQPDDFATYYCQQYNSHSWTFGQGTKVEIK |
| 55 | LC | QSIDNW |
| | CDR1 | |
| 56 | LC | KAS |
| | CDR2 | |
| 57 | LC | QQYNSHSWT |
| | CDR3 | |

TABLE 8

| SEQ | | |
|---|---|---|
| ID | | |
| NO: | | 07D09 |
| 58 | VH | QVQLVQSGAEVKKPGASVKVSCKASGYTFTGYYMHWV |
| | | RQAPGQGLEWMGWINPHSGGTNYAQKFQGRVTMTRDT |
| | | SISTAYMELSRLRSDDTAVYSCASGLGISAFDIWGQG |
| | | TMVTVSS |
| 59 | HC | GYTFTGYY |
| | CDR1 | |
| 60 | HC | INPHSGGT |
| | CDR2 | |
| 61 | HC | ASGLGISAFDI |
| | CDR3 | |
| 62 | VL | SYVLTQPPSVSVAPGQTARITCGGNNIGSKSVHWYQQ |
| | | KPGQAPVLVVYDDSDRPSGIPERFSGSNSGNTATLTI |
| | | SRVEAGDEADYYCQVWDSSSDHYVFGTGTKVTVL |
| 63 | LC | NIGSKS |
| | CDR1 | |
| 64 | LC | DDS |
| | CDR2 | |
| 65 | LC | QVWDSSSDHYV |
| | CDR3 | |

TABLE 9

| SEQ | | |
|---|---|---|
| ID | | |
| NO: | | 09L18 |
| 66 | VH | EVQLVESGGGLVKPGGSLRLSCAASGFTFSSYSMNWV |
| | | RQAPGKGLEWVSSIGSSSGYIFYADSVKGRFTISRDN |
| | | AKNSLYLQMNSLRAEDTAVYFCARDGGNFDYWGQGTL |
| | | VTVSS |
| 67 | HC | GFTFSSYS |
| | CDR1 | |
| 68 | HC | IGSSSGYI |
| | CDR2 | |
| 69 | HC | ARDGGNFDY |
| | CDR3 | |
| 70 | VL | QPVLTQPSSHSASSGASVRLTCMLSSGFSVGDFWIRW |
| | | YQQKPGNPPRYLLYYHSDSNKGQGSGVPSRFSGSNDA |
| | | SANAGILRISGLQPEDEADYYCGSWHTDSKTHWVFGG |
| | | GTKLTVL |
| 71 | LC | SGFSVGDFW |
| | CDR1 | |
| 72 | LC | YHSDSNK |
| | CDR2 | |
| 73 | LC | GSWHTDSKTHWV |
| | CDR3 | |

TABLE 10

| SEQ | | |
|---|---|---|
| ID | | |
| NO: | | 11A04 |
| 74 | VH | EVQLLESGGGLVQPGGSLRLSCAASGFTFNSSAMSWV |
| | | RQAPGKGLEWVSVISGSGGSTYYADSVKGHFTISRDN |
| | | SKNTLYLQMNSLRAEDTAVYYCAKKELGDWYFDLWGR |
| | | GTLVTVSS |
| 75 | HC | GFTFNSSA |
| | CDR1 | |
| 76 | HC | ISGSGGST |
| | CDR2 | |
| 77 | HC | AKKELGDWYFDL |
| | CDR3 | |
| 78 | VL | DIVMTQSPDSLAVSLGERATINCKSSQSVLYSSNNKN |
| | | YLAWYQQRPGQPPKLLIYWASTRESGVPDRFSGSGSG |
| | | TDFTLTISSLQAEDVAIYYCQQYYSIPPTFGQGTKVE |
| | | IK |
| 79 | LC | QSVLYSSNNKNY |
| | CDR1 | |
| 80 | LC | WAS |
| | CDR2 | |
| 81 | LC | QQYYSIPPT |
| | CDR3 | |

TABLE 11

| SEQ | | |
|---|---|---|
| ID | | |
| NO: | | 11G12 |
| 82 | VH | EVQLVESGGGLVKPGGSLRLSCAASGFTFSSYDMNWV |
| | | RQAPGKGLEWVSSISSSSSYIYYADSVKGRFTISRDN |
| | | AKNSLYLQMNSLRAEDTAVYYCARDSYDSSGYYLRPF |
| | | DYWGQGT |
| | | LVTVSS |
| 83 | HC | GFTFSSYD |
| | CDR1 | |
| 84 | HC | ISSSSSYI |
| | CDR2 | |
| 85 | HC | ARDSYDSSGYYLRPFDY |
| | CDR3 | |
| 86 | VL | DIQMTQSPSTLSASVGDRVTITCRASQSIDNWLAWYQ |
| | | QKPGKAPKLLIYKASSLESGVPSRFSGSGSGTEFILT |
| | | ISSLQPDDFATYYCQQYNSHSWTFGQGTKVEIK |
| 87 | LC | QSIDNW |
| | CDR1 | |
| 88 | LC | KAS |
| | CDR2 | |
| 89 | LC | QQYNSHSWT |
| | CDR3 | |

TABLE 12

| SEQ | | |
|---|---|---|
| ID | | |
| NO: | | 12K18 (kappa) |
| 90 | VH | EVQLVESGGGLVQPGGSLRLSCAASGFTFSSYWMHWV |
| | | RQAPGKGLVWVSRINSDGSSTSYADSVKGRFTISRDN |
| | | AKNTLYLQMNSLRAEDTAVYYCARGGSGSYFDYWGQG |
| | | TLVT |
| | | VSS |
| 91 | HC | GFTFSSYW |
| | CDR1 | |
| 92 | HC | INSDGSST |
| | CDR2 | |
| 93 | HC | ARGGSGSYFDY |
| | CDR3 | |
| 94 | VL | DVVMTQSPLSLPVTLGQPASVSCRSSQSLVYSDGNTY |
| | | LNWFQQRPGQSPRRLIYKVSNRDSGVPDRFSGSGSGT |
| | | DFTLKISRVEAEDVGVYYCMQGTHWPLTFGGGTKVEI |
| | | K |
| 95 | LC | QSLVYSDGNTY |
| | CDR1 | |
| 96 | LC | KVS |
| | CDR2 | |
| 97 | LC | MQGTHWPLT |
| | CDR3 | |

TABLE 13

| SEQ | | |
|---|---|---|
| ID | | |
| NO: | | 12K18 (lambda) |
| 98 | VH | EVQLVESGGGLVQPGGSLRLSCAASGFTFSSYWMHWV |
| | | RQAPGKGLVWVSRINSDGSSTSYADSVKGRFTISRDN |
| | | AKNTLYLQMNSLRAEDTAVYYCARGGSGSYFDYWGQG |
| | | TLVT |
| | | VSS |
| 99 | HC | GFTFSSYW |
| | CDR1 | |
| 100 | HC | INSDGSST |
| | CDR2 | |
| 101 | HC | ARGGSGSYFDY |
| | CDR3 | |
| 102 | VL | QSVLTQPPSVSGAPGQRVTISCTGSSSNIGAGFDVHW |
| | | YQQLPGTAPKLLIYFTNNRPSGVPDRFSGSKSGTSAS |
| | | LAITGLQAEDESDYYCQSYDSSLRWVFGGGTKLTVL |
| 103 | LC | SSNIGAGFD |
| | CDR1 | |
| 104 | LC | FTN |
| | CDR2 | |
| 105 | LC | QSYDSSLRWV |
| | CDR3 | |

An anti-MuSK antibody disclosed herein may be a monoclonal antibody, a humanized antibody, a chimeric antibody, a single chain antibody, a Fab fragment, a single-chain variable fragment (scFv), a recombinant antibody, a recombinant monoclonal antibody, an aptamer, a single-domain antibody (VHH, nanobody), or other MuSK-binding fragment or variant. In certain embodiments, an anti-MuSK antibody disclosed herein may comprise complementarity determining regions (CDRs) from a source (parental) antibody that have been grafted (fused) into a framework from a different type (class) of antibody and/or from a different organism than the parental antibody, in particular an acceptor human framework. In certain embodiments, an anti-MuSK antibody disclosed herein may comprise a framework in which amino acids have been substituted, mutated, or replaced in regions outside of the CDRs to influence properties such as antigen-binding or antibody structure, e.g., in the variable region framework surrounding the CDRs and/or in a constant region, in particular the Fc region. In certain embodiments, one or more of the CDRs have been substituted, mutated, or replaced. In certain embodiments, an anti-MuSK antibody disclosed herein may be a humanized anti-MuSK antibody variant. In preferred embodiments, the anti-MuSK antibody is a monoclonal antibody. In other preferred embodiments, the anti-MuSK antibody is a human or fully human monoclonal antibody.

The anti-MuSK antibodies of the present disclosure may further include a radiolabel. Examples of such radiolabels include ¹³¹I, ⁹⁰Y ²¹²Bi, ¹⁸⁶Re, ²²¹At, ^{99m}Tc, and mixtures thereof. However, other radiolabels known in the art may also be utilized.

As described herein, the present anti-MuSK antibody may serve as a drug delivery system enabling delivery of a peptide or drug to the NMJ without significantly impacting endogenous MuSK function.

The anti-MuSK antibody may be prepared by the methods described in the instant examples or any other suitable method known to a skilled artisan.

### The PRAD Peptide

In a second aspect, the present disclosure describes a proline-rich attachment domain (PRAD) peptide comprising a core sequence that is optionally flanked by a first sequence and/or a second sequence. The core sequence is selected from the group consisting of LLTPPPPPLFPPPFF (SEQ ID NO:106), LLMPPPPPLFPPPFF (SEQ ID NO:107), and LLTPPPPPMFPPPFF (SEQ ID NO:108). The core sequence is synthetic and prepared by standard methods known to a skilled artisan. The first sequence, when present, is located on the N-terminus of the core sequence and the second sequence, when present, is located on the C-terminus of the core sequence. In embodiments, the PRAD peptide is conjugated to the antibody or binding means via either cysteine-based chemical approaches or chemo-enzymatic methods. The first sequence consists of one (1) to six (6) amino acids. The second sequence consists of one (1) to eight (8) amino acids. The PRAD peptide may consist of or comprise 15, 16, 17, 18, 19, 20, 21, 22, 23, 24, 25, 26, 27, 28, or 29 amino acids. The PRAD peptide may comprise or consist of the core sequence and a first sequence comprising or consisting of 1, 2, 3, 4, 5, or 6 amino acids. The PRAD peptide may comprise, consist of, or consist essentially of the core sequence and a second sequence comprising or consisting of 1, 2, 3, 4, 5, 6, 7, or 8 amino acids. The PRAD peptide may comprise or consist of the core sequence, a first sequence comprising or consisting of 1 amino acid, and a second sequence comprising or consisting of 1, 2, 3, 4, 5, 6, 7, or 8 amino acids. The PRAD peptide may comprise or consist of the core sequence, a first sequence comprising or consisting of 2 amino acids, and a second sequence comprising or consisting of 1, 2, 3, 4, 5, 6, 7, or 8 amino acids. The PRAD peptide may comprise or consist of the core sequence, a first sequence comprising or consisting of 3 amino acids, and a second sequence comprising or consisting of 1, 2, 3, 4, 5, 6, 7, or 8 amino acids. The PRAD peptide may comprise or consist of the core sequence, a first sequence comprising or consisting of 4 amino acids, and a second sequence comprising or consisting of 1, 2, 3, 4, 5, 6, 7, or 8 amino acids. The PRAD peptide may comprise or consist of the core sequence, a first sequence comprising or consisting of 5 amino acids, and a second sequence comprising or consisting of 1, 2, 3, 4, 5, 6, 7, or 8 amino acids. The PRAD peptide may comprise or consist of the core sequence, a first sequence comprising or consisting of 6 amino acids, and a second sequence comprising or consisting of 1, 2, 3, 4, 5, 6, 7, or 8 amino acids. The PRAD peptide may comprise or consist of the core sequence, a second sequence comprising or consisting of 1 amino acid, and a first sequence comprising or consisting of 1, 2, 3, 4, 5, or 6 amino acids. The PRAD peptide may comprise or consist of the core sequence, a second sequence comprising or consisting of 2 amino acids, and a first sequence comprising or consisting of 1, 2, 3, 4, 5, or 6 amino acids. The PRAD peptide may comprise or consist of the core sequence, a second sequence comprising or consisting of 3 amino acids, and a first sequence comprising or consisting of 1, 2, 3, 4, 5, or 6 amino acids. The PRAD peptide may comprise or consist of the core sequence, a second sequence comprising or consisting of 4 amino acids, and a first sequence comprising or consisting of 1, 2, 3, 4, 5, or 6 amino acids. The PRAD peptide may comprise or consist of the core sequence, a second sequence comprising or consisting of 5 amino acids, and a first sequence comprising or consisting of 1, 2, 3, 4, 5, or 6 amino acids. The PRAD peptide may comprise or consist of the core sequence, a second sequence comprising or consisting of 6 amino acids, and a first sequence comprising or consisting of 1, 2, 3, 4, 5, or 6 amino acids. The PRAD peptide may comprise or consist of the core sequence, a second sequence comprising or consisting of 7 amino acids, and a first sequence comprising or consisting of 1, 2, 3, 4, 5, or 6 amino acids. The PRAD peptide may comprise or consist of the core sequence, a second sequence comprising or consisting of 8 amino acids, and a first sequence comprising or consisting of 1, 2, 3, 4, 5, or 6 amino acids. The PRAD peptide may comprise a core sequence and: a first sequence with 1 amino acid, a first sequence with 2 amino acids, a first sequence with 3 amino acids, a first sequence with 4 amino acids, a first sequence with 5 amino acids, a first sequence with 6 amino acids, a second sequence with 1 amino acid, a second sequence with 2 amino acids, a second sequence with 3 amino acids, a second sequence with 4 amino acids, a second sequence with 5 amino acids, a second sequence with 6 amino acids, a second sequence with 7 amino acids, a second sequence with 8 amino acids, a first sequence with 1 amino acid and a second sequence with 1 amino acid, a first sequence with 1 amino acid and a second sequence with 2 amino acids, a first sequence with 1 amino acid and a second sequence with 3 amino acids, a first sequence with 1 amino acid and a second sequence with 4 amino acids, a first sequence with 1 amino acid and a second sequence with 5 amino acids, a first sequence with 1 amino acid and a second sequence with 6 amino acids, a first sequence with 1 amino acid and a second sequence with 7 amino acids, a first sequence with 1 amino acid and a second sequence with 8 amino acids, a first sequence with 2 amino acids and a second sequence with 1 amino acid, a first sequence with 2 amino acids and a second sequence with 2 amino acids, a first sequence with 2 amino acids and a second sequence with 3 amino acids, a first sequence with 2 amino acids and a second sequence with 4 amino acids, a first sequence with 2 amino acids and a second sequence with 5 amino acids, a first sequence with 2 amino acids and a second sequence with 6 amino acids, a first sequence with 2 amino acids and a second sequence with 7 amino acids, a first sequence with 2 amino acids and a second sequence with 8 amino acids, a first sequence with 3 amino acids and a second sequence with 1 amino acid, a first sequence with 3 amino acids and a second sequence with 2 amino acids, a first sequence with 3 amino acids and a second sequence with 3 amino acids, a first sequence with 3 amino acids and a second sequence with 4 amino acids, a first sequence with 3 amino acids and a second sequence with 5 amino acids, a first sequence with 3 amino acids and a second sequence with 6 amino acids, a first sequence with 3 amino acids and a second sequence with 7 amino acids, a first sequence with 3 amino acids and a second sequence with 8 amino acids, a first sequence with 3 amino acids and a second sequence with 1 amino acid, a first sequence with 3 amino acids and a second sequence with 2 amino acids, a first sequence with 3 amino acids and a second sequence with 3 amino acids, a first sequence with 3 amino acids and a second sequence with 4 amino acids, a first sequence with 3 amino acids and a second sequence with 5 amino acids, a first sequence with 3 amino acids and a second sequence with 6 amino acids, a first sequence with 3 amino acids and a second sequence with 7 amino acids, a first sequence with 3 amino acids and a second sequence with 8 amino acids, a first sequence with 4 amino acids and a second sequence with 1 amino acid, a first sequence with 4 amino acids and a second sequence with 2 amino acids, a first sequence with 4 amino acids and a second sequence with 3 amino acids, a first sequence with 4 amino acids and a second sequence with 4 amino acids, a first sequence with 4 amino acids and a second sequence with 5 amino acids, a first sequence with 4 amino acids and a second sequence with 6 amino acids, a first sequence with 4 amino acids and a second sequence with 7 amino acids, a first sequence with 4 amino acids and a second sequence with 8 amino acids, a first sequence with 5 amino acids and a second sequence with 1 amino acid, a first sequence with 5 amino acids and a second sequence with 2 amino acids, a first sequence with 5 amino acids and a second sequence with 3 amino acids, a first sequence with 5 amino acids and a second sequence with 4 amino acids, a first sequence with 5 amino acids and a second sequence with 5 amino acids, a first sequence with 5 amino acids and a second sequence with 6 amino acids, a first sequence with 5 amino acids and a second sequence with 7 amino acids, a first sequence with 5 amino acids and a second sequence with 8 amino acids, a first sequence with 6 amino acids and a second sequence with 1 amino acid, a first sequence with 6 amino acids and a second sequence with 2 amino acids, a first sequence with 6 amino acids and a second sequence with 3 amino acids, a first sequence with 6 amino acids and a second sequence with 4 amino acids, a first sequence with 6 amino acids and a second sequence with 5 amino acids, a first sequence with 6 amino acids and a second sequence with 6 amino acids, a first sequence with 6 amino acids and a second sequence with 7 amino acids, or a first sequence with 6 amino acids and a second sequence with 8 amino acids.

The PRAD peptide may be of formula (1): X₁-X₂-X₃-X₄-X₅-X₆-CORE-X₇-X₈-X₉-X₁₀-X₁₁-X₁₂-X₁₃-X₁₄; wherein X₁ is absent or any natural human amino acid, preferably C; X₂ is absent or any natural human amino acid, preferably C; X₃ is absent or any natural human amino acid; X₄ is absent or any natural human amino acid; X₅ is absent or any natural human amino acid; X₆ is absent or any natural human amino acid; X₇ is absent or any natural human amino acid, preferably R, G, or S; X₃ is absent or any natural human amino acid, preferably R, G, or S; X₉ is absent or any natural human amino acid, preferably R, G, or S; X₁₀ is absent or any natural human amino acid, preferably R, G, or S; X₁₁ is absent or any natural human amino acid, preferably R, G, or S; X₁₂ is absent or any natural human amino acid, preferably R, G, or S; X₁₃ is absent or any natural human amino acid; X₁₄ is absent or any natural human amino acid, and CORE is selected from the group of SEQ ID NO. 106, 107 and 108, preferably SEQ ID NO. 106.

Exemplary PRAD peptides comprising a first sequence and a core include CCLLTPPPPPLFPPPFF (SEQ ID NO:109) (“PRADshort”). Exemplary PRAD peptides comprising a first sequence, a core sequence, and a second sequence include CCLLTPPPPPLFPPPFFRGGRS (SEQ ID NO:110) (“PRADlong”).

In embodiments, the PRAD peptide binds or recruits an endogenous protein or compound. For example, the PRAD peptide may bind to AChE and/or recruits AChE to the NMJ.

Two, three, four, five, six, seven, or eight PRAD peptides may be associated with the anti-MuSK antibody or binding means. In preferred embodiments, two (2), four (4), six (6), or eight (8) PRAD peptides are conjugated to each anti-MuSK antibody or binding means.

The PRAD peptide can be synthesized and conjugated to the anti-MuSK antibody or binding means by conventional techniques known to those skilled in the art. For example, two or more identical PRAD peptides can be synthesized in tandem via a linker. This linker can be non-repetitive, flexible or repetitive (e.g., (G4S)₃ or (G3S)₄). The PRAD peptide can be conjugated to the anti-MuSK antibody or binding means via cysteine-based chemical conjugation methods, particularly when the peptide-to-antibody ratio (PAR) is 4 or higher. A chemo-enzymatic approach could also be used where the linking peptide can further comprise one or more reactive groups, such as dibenzocyclooctyne (DBCO), for use in click chemistry.

### Antibody Peptide Conjugate (APC)

In a third aspect, the present disclosure describes an antibody peptide conjugate comprising a means for binding MuSK or an anti-MuSK anbody as described herein and a PRAD peptide as described herein.

The antibody peptide conjugate may be prepared by the methods described in the instant examples or any other suitable method known to a skilled artisan, such as the methods described by Sadiki et al. (Antibody Therapeutics (2020) 3:4: 271-284), which is hereby incorporated by reference in its entirety.

NMJ-targeted Drug Delivery Platform In a fourth aspect, the present disclosure describes an antibody-drug conjugate, optionally further comprising a PRAD peptide. In these embodiments, the anti-MuSK antibody serves as an NMJ-targeted drug delivery platform, which can deliver the drug to NMJ, increase the local drug concentration on NMJ, and lower the effective dose of the drug required to achieve the same therapeutic benefit and avoid or reduce undesirable side effects associated with a higher dose.

The drug may be conjugated to an anti-MuSK antibody described herein utilizing methods well known to a skilled artisan. For example, the drug may be conjugated to the anti-MuSK antibody via a linker. In embodiments, the linker is used in site-specific chemo-enzymatic or chemical conjugation. The drug may be any biologically active compound that exhibits one or more beneficial effects in the NMJ. For example, the drug may be an acetylcholine receptor inhibitor, an acetylcholinesterase inhibitor, or an adrenergic agonist.

In a fifth aspect, the antibody peptide conjugate of the present disclosure is capable of anchoring acetylcholinesterase (AChE) to the NMJ.

In a sixth aspect, the antibody peptide conjugate of the present disclosure is capable of enhancing or replacing the function of CoIQ in the NMJ.

### Examples

Example 1. Generation and Identification of Anti-MuSK antibodies. AlivaMab Mice, a transgenic mouse line producing antibodies with fully-human F(ab′)2 and mouse CH2 and CH3 domains wherein the fully human F(ab′)2 comprises human VH and VL, and human CL and CH1, was utilized from Ablexis, LLC (San Diego, CA) and used in immunization and generation of antibodies of the present disclosure. Nine Kappa-Lambda AlivaMab Mice (3× AMM-XKL1, 3× XKL2, and 3× XKL3) (from AlivaMab Biologics, LLC (formerly AlivaMab Discovery Services, LLC, San Diego, CA)) were primed with a mixture of DNA expressing full-length human MuSK conjugated with super T cell epitope (TCE) (pADS38-huMuSK FL-TCE) and DNA expressing full-length mouse MuSK conjugated with TCE (pADS38-muMuSK FL-TCE). The mice were then boosted 6 times with a mixture of recombinant proteins and recombinant proteins conjugated with TCE, including huMuSK Ig3-Fz (recombinant protein containing Ig3 and Fz domains of human MuSK), muMuSK Ig3-Fz (recombinant protein containing Ig3 and Fz domains of mouse MuSK), huMuSK Ig3-Fz-TCE conjugate, and muMuSK Ig3-Fz-TCE conjugate. Before the final boost, plasma titers were checked to ensure they were sufficient. Plasma titers were determined by fluorescence-activated cell sorting (FACS) against HEK293T cell lines stably expressing human, mouse, or cynomolgus monkey MuSK (293T: huMuSK, 293T: muMuSK, or 293T: cyMuSK) and by enzyme-linked immunosorbent assay (ELISA) against recombinant protein containing the full extracellular domain (ECD) of human or mouse MuSK (huMuSK ECD or muMuSK ECD). Following the final boost, B cells were isolated and enriched from spleens and lymph nodes, and subsequently used for electrofusion to generate hybridomas.

Hybridomas were plated into 384-well plates at a density of 1-2 hybridomas per well. Hybridoma supernatants were screened by FACS against 293T: huMuSK, 293T: muMuSK, or parental 293T and by ELISA against huMuSK Ig3-Fz or muMuSK Ig3-Fz. 376 hybridomas were selected based on the FACS and ELISA results. Some of those hybridomas showed strong binding against 293T: huMuSK and 293T: muMuSK in FACS and strong binding against huMuSK Ig3-Fz and muMuSK Ig3-Fz in ELISA. Other hybridomas only showed strong binding against 293T: huMuSK and 293T: muMuSK in FACS.

The 376 hybridomas were then moved forward to a confirmation screen, which includes FACS against 293T: huMuSK, 293T: muMuSK, or 293T: cyMuSK and ELISA against huMuSK ECD or muMuSK ECD. 221 hybridomas were selected in the confirmation screen. 215 of them had high affinity with human, mouse, and cynomolgus monkey MuSK in FACS (hu/mu/cy MuSK triple binders). 6 of them had high affinity with human and cynomolgus monkey MuSK but low affinity with mouse MuSK in FACS and ELISA (hu/cy MuSK double binders). The selected hybridomas were then expanded. The saturated supernatants (supes) from the hybridomas were harvested for functional assays to identify MuSK agonist hybridomas.

Example 2. Identification of Non-Agonistic MuSK Hybridomas. The aim of this example is to identify an anti-MuSK antibody backbone that does not affect endogenous MuSK function. Anti-MuSK antibodies tend to dimerize MuSK on membrane and activate MuSK. In order to screen for non-agonistic MuSK antibody in large scale, a cell-based MuSK activation assay was developed. A Ba/F3 cell pool stably expressing a chimeric receptor containing extracellular domain of human MuSK and transmembrane and intracellular domains of human Thrombopoietin receptor (c-MpI) was generated (Ba/F3: MuSK-cMpI). The activation of this chimeric receptor by MuSK agonist antibody causes the dimerization of the receptor activating the signaling pathway downstream of c-MpI and induces cell proliferation, which can be measured by proliferation assay. We tested the saturated supes from 215 hybridomas selected from confirmation screen (hu/mu/cy MuSK triple binders) and saturated supes containing antibodies not targeting MuSK (Negative Control) in MuSK activation assay. Ba/F3: MuSK-cMpI cells were maintained in the culture medium containing RPMI-1640 (Gibco, 11875093), 10% FBS (Gibco, 10082-147), 1% Pen Strep (Gibco, 15140-122) and 5 ng/mL mouse IL-3 (Sino Biological, 51066-MNAH) in a CO2 incubator (5% CO2) at 37° C. Cells were washed once with RPMI-1640 and transferred to IL-3 free culture medium containing RPMI-1640, 10% FBS, and 1% anti-anti and incubated for 1 day. Cells were washed once with RPMI-1640, and treated for 3 days with 2% saturated supe in IL-3 free medium in a 96-well culture plate (5000 cells in 100 μL medium per well). At the end of the treatment, cell proliferation was measured using CellTiter-Glo® Luminescent Cell Viability Assay kit (Promega, G7571) according to the manufacturer's instruction. Luminescence was measured in CLARIOstar□ Plus Multi-mode Plate Reader (BMG Labtech). The Relative Light Unit (RLU) of all treated cells was listed in Table 14, wheret he values are the average RLU of two wells under the same treatment. Among the 215 hybridomas, 11 induced weak proliferation with the average RLU lower than 116,230, and were identified as non-agonistic MuSK binders. Their luminescence intensity in the cell viability assay was listed in Table 14. In contrast, some hybridomas induced strong proliferation with RLU values higher than 1,000,000, and were categorized as anti-MuSK agonists. Three of these were listed in Table 14 as positive controls. The average RLU value of Negative Controls was around 11,000.

TABLE 14
Average RLU in cell viability assay of
Ba/F3: MuSK-cMpl cells treated with 2%
saturated supes or 1.5 μg/mL rfmAb-1
for 3 days.

| ID | Average RLU |
|---|---|
| 02C13 | 116230 |
| 02O12 | 37896 |
| 05D18 | 90004 |
| 06B20 | 69858 |
| 06E17 | 95755 |
| 07A13 | 36125 |
| 07D09 | 26851 |
| 09L18 | 35834 |
| 11A04 | 56360 |
| 11G12 | 73129 |
| 12K18 | 76533 |
| 09G09 | 2002594 |
| (Positive | |
| Control-1) | |
| 04N24 | 1680767 |
| (Positive | |
| Control-2) | |
| 02E22 | 1292679 |
| (Positive | |
| Control-3) | |
| Negative | 10177 |
| Control-1 | |
| Negative | 11462 |
| Control-2 | |
| Negative | 11759 |
| Control-3 | |

Example 3. Characterization of Anti-MuSK antibodies. The 11 non-agonistic hybridoma hits, namely 02C13A, 02012A, 05D18A, 06B20A, 06E17A, 07A13A, 07D09A, 09L18A, 11A04A, 11G12A, and 12K18A, were successfully subcloned and developed into monoclonal antibodies (mAbs). The mAbs were produced in human F(ab′)2/mouse CH2-CH3 format, and their variable regions were sequenced. See, Tables 1-13.

Affinity Analysis. The affinity of the 11 mAbs to human, cynomulgus monkey, and mouse MuSK were quantified with FACS KD analysis. Serial dilution of mAb (initial concentration 10 nM, 5-fold dilutions, 8-point dilution series in duplicate) were added to 293T: huMuSK, 293T: cyMuSK, or 293T: muMuSK cells seeded in 96-well plates (50,000 cells per well). Cells were incubated at 4° C. overnight. After washing with FACS buffer twice, cells were incubated with Goat anti-Mouse IgG (H+L) Cross-Adsorbed Secondary Antibody, Alexa Fluor™ 647 (Thermo Fisher, A32728) diluted at 1:2000 and Helix Blue diluted at 1:2000 at 4° C. for 30 min. After washing with FACS buffer twice, cells were subjected to iQue3 Screener for FACS analysis. Geometric mean fluorescence intensity (gMFI) of A647 was plotted in Graphpad Prism for EC50 calculation. The results of FACS analysis were listed in Table 15. Eight of the 11 mAbs showed high affinity with human/cyno monkey/mouse MuSK, suggesting good cross reactivity to three species of MuSK. 09L18A and 11A04A showed relatively high affinity with human/cyno monkey MuSK, but low affinity with mouse MuSK. 07A13A showed relatively low affinity with MuSK of all three species.

TABLE 15
FACS KD of 11 anti-MuSK mAbs against 293T:
huMuSK, 293T: cyMuSK, and 293T: muMuSK.

| | 293T: huMuSK | | | 293T: cyMuSK | | | 293T: muMuSK | | |
|---|---|---|---|---|---|---|---|---|---|
| | gMFI | EC50 | | gMFI | EC50 | | gMFI | EC50 | |
| ID | max | (nM) | R² | max | (nM) | R² | max | (nM) | R² |
| 02C13A | 2926869 | 0.11 | 0.98 | 2480209 | 0.15 | 1.00 | 634961 | 0.13 | 0.96 |
| 02O12A | 1171536 | 0.04 | 0.97 | 691278 | 0.04 | 0.99 | 400496 | 0.01 | 0.92 |
| 05D18A | 2908517 | 0.07 | 0.98 | 2109145 | 0.10 | 1.00 | 875106 | 0.17 | 1.00 |
| 06B20A | 1301447 | 0.11 | 1.00 | 1009391 | 0.14 | 0.98 | 732144 | 0.12 | 0.95 |
| 06E17A | 1089661 | 0.20 | 1.00 | 672209 | 0.26 | 1.00 | 347470 | 0.03 | 0.98 |
| 07A13A | 875424 | 1.45 | 1.00 | 535799 | 1.34 | 0.99 | 428618 | 0.72 | 1.00 |
| 07D09A | 1322564 | 0.31 | 0.99 | 909510 | 0.20 | 1.00 | 1258517 | 0.19 | 0.93 |
| 09L18A | 2164597 | 0.08 | 0.99 | 1199244 | 0.06 | 0.98 | 441653 | 3.46 | 1.00 |
| 11A04A | 4045541 | 0.10 | 0.99 | 1459529 | 0.11 | 1.00 | 291241 | 9.48 | 1.00 |
| 11G12A | 1655975 | 0.13 | 1.00 | 740271 | 0.11 | 0.97 | 918423 | 0.09 | 0.99 |
| 12K18A | 1815691 | 0.09 | 1.00 | 981801 | 0.07 | 0.99 | 750390 | 0.13 | 0.98 |

Epitope Binding. Next, the same eleven (11) mAbs were subjected to ELISA analysis to examine their binding sites on human MuSK. High-binding 96-well plates (Corning, 9018) were coated with 100 μL per well of huMuSK ECD, huMuSK Fz, or huMuSK Ig3-Fz (2 μg/mL in PBS) overnight at 4° C. After 3 washes with 1×Phosphate-Buffered Saline, 0.1% Tween 20 (PBST), each plate was blocked with 1% BSA in PBS for 1 h at room temperature (RT). Diluents of anti-MuSK antibodies (3-fold serial dilutions with concentrations in the range of 0-100 nM) were added to plates. The plate was incubated for 1 h at RT and washed 3 times with PBST. The plate was then incubated with HRP conjugated goat anti-mouse IgG (Thermo Fisher Scientific, G-21040) 1:5000 diluted in 1% BSA in PBS for 1 h at RT and washed 3 times. 3,3′, 5,5′-tetramethylbenzidine (TMB) Stabilized Chromogen substrate solution (Thermo Fisher Scientific, SB02) was added to the plate, and the reaction was stopped by adding 1M sulfuric acid. The absorbance at 450 nm was measured in a plate reader. The EC50 of each antibody against Fz, Ig3-Fz, and ECD of human MuSK was listed in Table 16. Based on these data, the binding region of each antibody on MuSK was predicted and shown in Table 16.

TABLE 16
EC50 of anti-MuSK antibodies against Fz,
Ig3-Fz, and ECD of human MuSK in ELISA

| | Fz EC50 | Ig3-Fz EC50 | ECD EC50 | Predicted binding |
|---|---|---|---|---|
| ID | (nM) | (nM) | (nM) | region |
| 02C13A | 0.072 | 0.081 | 0.093 | Fz |
| 02O12A | N.D. | 0.065 | 0.061 | Ig3 |
| 05D18A | 0.062 | 0.095 | 0.118 | Fz |
| 06B20A | 0.189 | 0.084 | 0.089 | Fz |
| 06E17A | 0.076 | 0.071 | 0.075 | Fz |
| 07A13A | 1.766 | 0.146 | 0.105 | Fz |
| 07D09A | 0.048 | 0.032 | 0.040 | Fz |
| 09L18A | 0.099 | 0.085 | 0.093 | Fz |
| 11A04A | 0.145 | 0.069 | 0.076 | Fz |
| 11G12A | 0.140 | 0.070 | 0.085 | Fz |
| 12K18A | 0.183 | 0.116 | 0.143 | Fz |

As detailed in Table 16, 02012A had no affinity with Fz but demonstrated high affinity with Ig3-Fz and ECD, suggesting it likely binds to the Ig3 domain of MuSK. All other antibodies had high affinity with Fz, Ig3-Fz, and ECD, indicating they likely bind to the Fz domain of MuSK.

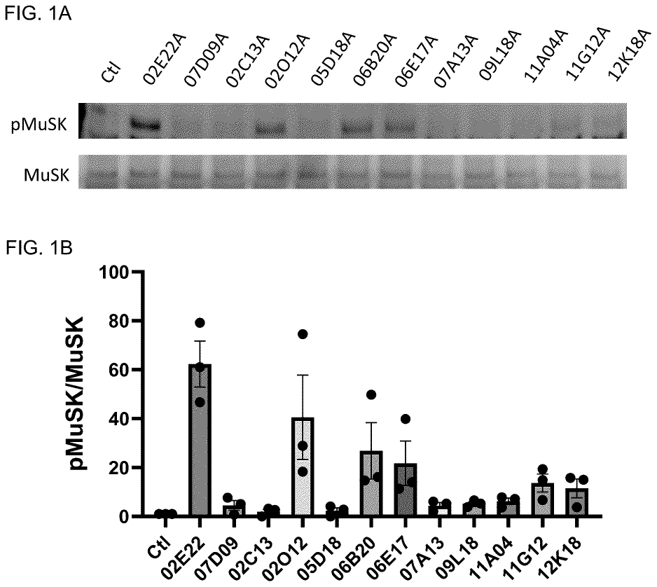

Activation of MuSK. To test whether the 11 MuSK-binding mAbs affect MuSK activity in muscle cells, MuSK phosphorylation (indicating MuSK activation) in C2C12 myotubes treated with anti-MuSK antibody was examined. First, C2C12 mouse myoblast cells (ATCC, CRL-1772) were differentiated into myotubes. 2.5×105 C2C12 cells were cultured in a 60 mm petri dish for 3 days in DMEM (Gibco, 11995-040) with 10% FBS. The cells were then differentiated by switching to differentiation medium containing DMEM (without sodium pyruvate) (Gibco, 31053-028) with 2% horse serum (Gibco, 26050-088) and incubation for 2 days. Next, differentiated C2C12 was treated with 10 nM anti-MuSK antibody for 30 min. After the treatment, cells were immediately lysed in 500 μL per dish of Pierce™ IP Lysis Buffer (Thermo Scientific, 87787) plus Halt™ Protease and Phosphatase Inhibitor Cocktail (Thermo Scientific, 78444). Cell lysate was centrifuged at 150,000 g for 5 min at 4° C. to remove any pellet. MuSK was immunoprecipitated from cell lysate with a human anti-MuSK monoclonal antibody rfmAb-1 (hu-rfmAb-1). The immunoprecipitation was conducted using Dynabeads™ Protein G Immunoprecipitation Kit (Invirogen, 10007D) following manufacture's instruction. 5 μg hu-rfmAb-1 attached to 50 μL dynabeads was added to 500 μL cell lysate. Finally, immunoprecipitated samples were processed for Western Blot (WB). Samples were loaded into NuPAGE™ Bis-Tris 4-12% Mini Protein Gels (Invitrogen, NP0323BOX) and run at 200V for 35 min. Proteins were transferred to nitrocellulose membrane (Invitrogen, I123001). The membrane was blocked with 1× TRIS-buffered saline (TBST) with 5% BSA for 1 h at RT and probed with an anti-phosphotyrosine antibody (EMD Millipore, 05-321) diluted 1:1000 in TBST with 5% BSA or with an anti-MuSK polyclonal antibody (R&D Systems, AF562) diluted 1:1000 in TBST with 5% BSA overnight at 4° C. After 3 washes in TBST, the membrane was incubated with goat anti-mouse IgG, HRP (Invitrogen, G21040) or donkey anti-goat IgG, HRP (Invitrogen, A15999) for 2 h at RT. After 3 washes in TBST, the protein bands on membrane were revealed with SuperSignal™ West Dura Extended Duration Substrate (Thermo Scientific, 34076). The membrane was imaged under Western Blot Imaging System Azure C600 (Azure Biosystems, AZI600-01). The result of WB was shown in FIG. 1. The ratios of phospho-MuSK to total MuSK indicating levels of MuSK phosphorylation/activation were analyzed. As a positive control, an anti-MuSK agonistic antibody 02E22A significantly enhanced MuSK phosphorylation. 8 out of 11 anti-MuSK mAbs tested has little or no effect on MuSK phosphorylation. 3 mAbs, namely 02012A, 06E20A, and 06E17A, increased MuSK phosphorylation, suggesting that they may activate MuSK in muscle cells.

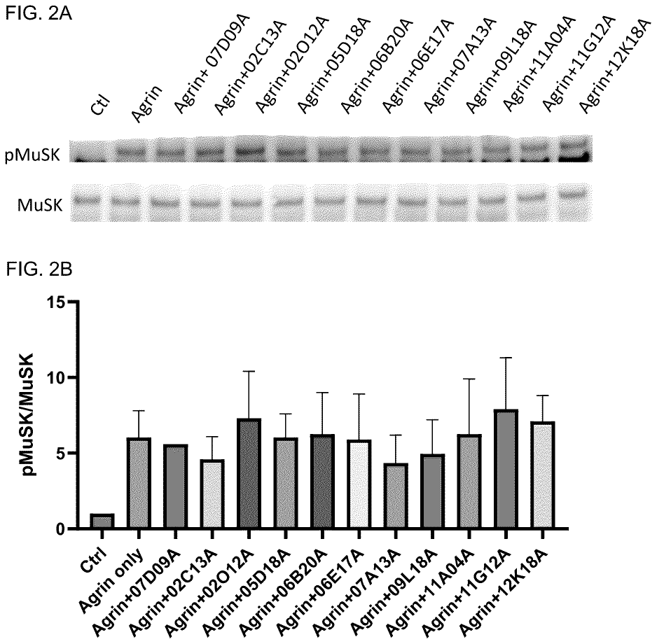

Agrin is an endogenous agonist of MuSK. It binds to MuSK-LRP4 complex and induces MuSK activation on neuromuscular junction. To test if the anti-MuSK mAb disrupts agrin induced MuSK activation, differentiated C2C12 cells were pre-treated with 10 nM anti-MuSK antibody for 1 h, and then treated with 1 nM rat Agrin (R&D systems, 550-AG-100) in the presence of anti-MuSK antibody. Cells were lysed and levels of MuSK phosphorylation was measured by WB as described above. The result of WB was shown in FIG. 2. Agrin induced strong MuSK phosphorylation in C2C12 myotubes. None of the 11 mAbs affects agrin-induced MuSK phosphorylation, suggesting that their binding with MuSK did not disrupt the activation of MuSK by agrin.

Based on the data, 02C13A and 07D09A exhibited high affinity for huMuSK, cyMuSK, and muMuSK, with little impact on MuSK activity or agrin-mediated MuSK activation. These antibodies also performed well in expression and purification implicating good developability (data not shown). Thus, 02C13A and 07D09A were advanced to serve as the backbones to generate anti-MuSK PRAD APCs.

Example 4. Generation of mu07D09A-PRAD APCs. For initial proof of concept studies, a random coupling strategy was employed to generate anti-MuSK-PRAD peptide conjugates. 07D09A was formatted into a mouse IgG2a lambda antibody with L234A, L235A, and P329G (LALAPG) mutations in the Fc region to eliminate effector function.

Two PRAD peptides were synthesized by Innopep, Inc. with >90% purity: A 17-amino acid peptide (CCLLTPPPPPLFPPPFF), referred to as PRADshort, and a 22-amino acid peptide (CCLLTPPPPPLFPPPFFRGGRS), referred to as PRADlong.

Random coupling of mu07D09A with PRAD peptides was performed using the PerKit™ Antibody Peptide Conjugation Kit (CellMosaic, Inc. Cat #CM32402x3). The process involved coupling N-maleimidopropyl-oxysuccinimide ester (BMPS), a bifunctional crosslinker with the antibody to introduce maleimide groups via succinimide reactions with the primary amine groups of the antibody, which subsequently reacted with the cysteine residues on the PRAD peptides, forming conjugates. Both PRADshort and PRADlong were conjugated to mu07D09 following the manufacturer's instructions, with a peptide-to-antibody molar ratio of 30:1.

The final conjugation yields were approximately 50% for the mu07D09A-PRADshort and 67% for the mu07D09A-PRADlong.

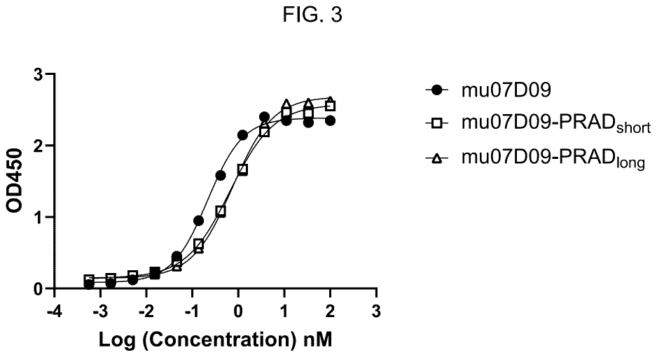

Example 5. In-vitro characterization of mu07D09A-PRAD APCs. To test whether mu07D09A-PRADshort and mu07D09A-PRADlong could still bind MuSK, an ELISA assay against huMuSK ECD was conducted. High-binding 96-well plates (Corning, 9018) were coated with 100 μL per well of huMuSK ECD (2 μg/mL in PBS) overnight at 4° C. After 3 washes with PBST, each plate was blocked with 1% BSA in PBS for 1 h at room temperature (RT). Diluents of mu07D09A, mu07D09A-PRADshort and mu07D09A-PRADlong (3-fold serial dilutions with concentrations in the range of 0-100 nM) were added to plates. The plate was incubated for 1 h at RT and washed 3 times with PBST. The plate was then incubated with HRP conjugated goat anti-mouse IgG (Thermo Fisher Scientific, G-21040) 1:5000 diluted in 1% BSA in PBS for 1 h at RT and washed 3 times. 3,3′, 5,5′-tetramethylbenzidine (TMB) Stabilized Chromogen substrate solution (Thermo Fisher Scientific, SB02) was added to the plate, and the reaction was stopped by adding 1M sulfuric acid. The absorbance at 450 nm was measured in a plate reader. The ELISA results are shown in FIG. 3. The EC50 of mu07D09A, mu07D09A-PRADshort and mu07D09A-PRADlong was 0.21, 0.66, and 0.74 nM, respectively. The affinity of mu07D09A-PRAD APC with huMuSK ECD was slightly lower than that of the parental antibody mu07D09A.

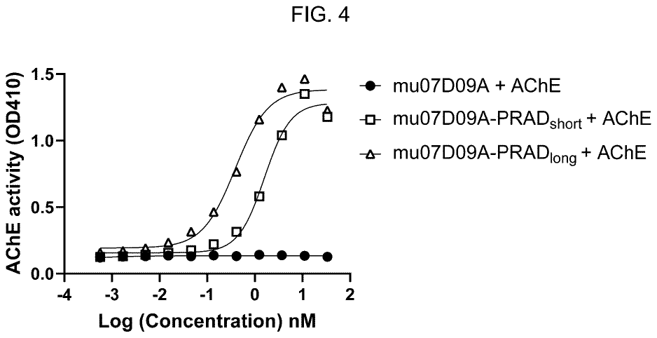

To test whether mu07D09A-PRADshort and mu07D09A-PRADlong could bind both MuSK and AChE, an AChE binding assay was developed. Purified recombinant human AChE (6 μg/mL or 88 nM or 16.5 U/mL, made in house) was mixed with serial dilutions (3-fold serial dilutions with concentrations in the range of 0-5 μg/mL (0-33 nM)) of mu07D09A, mu07D09A-PRADshort or mu07D09A-PRADlong overnight at 4° C. In the meantime, a high-binding 96-well plate (Corning, 9018) was coated with 100 μL per well of huMuSK ECD (2 μg/mL in PBS) overnight at 4° C. After 3 washes with PBST, the plate was blocked with 1% BSA in PBS for 1 h at RT. The antibody-AChE mixture (100 μL per well) was added to the plate and incubated for 2 h on shaker at 4° C. After 3 washes with PBST, the AChE activity on the plate was measured by Acetylcholinesterase Assay Kit (Abcam, ab138871) according to manufacturer's instruction. The AChE binding assay results are shown in FIG. 4. AChE activity was detected on the plate when mixed with mu07D09A-PRADshort or mu07D09A-PRADlong but not detected when mixed with mu07D09A, suggesting that mu07D09A-PRAD APCs recruit AChE to MuSK. Based on our knowledge, this is the first molecule other than natural CoIQ protein that is capable of binding both AChE and MuSK and couple AChE to MuSK. The EC50 of mu07D09A-PRADshort and mu07D09A-PRADlong was 1.59 and 0.39 nM, respectively. mu07D09A-PRADlong demonstrated higher AChE recruiting efficiency than mu07D09A-PRADshort, and therefore, PRADlong was used to generate human anti-MuSK antibody-PRAD conjugate.

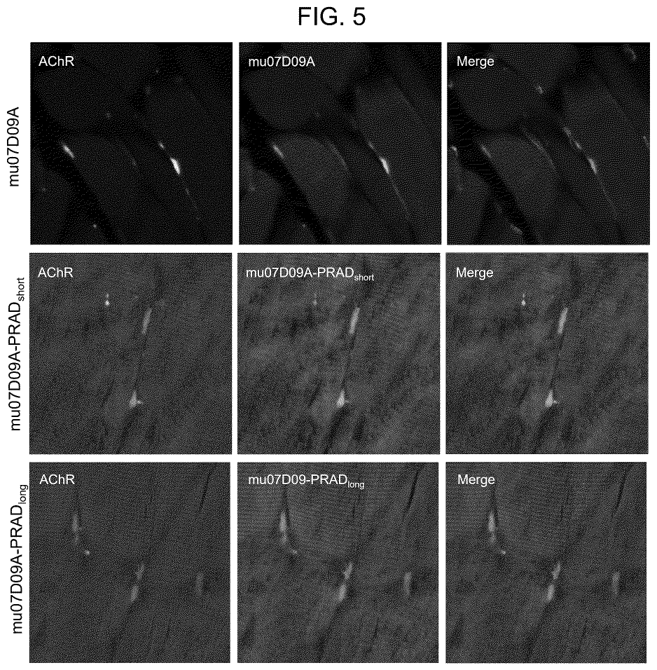

Example 6. ex-vivo functional study of mu07D09A-PRAD. Mu07D09A-PRAD binding to NMJ on human skeletal muscle sections. MuSK is expressed on NMJ. To examine whether the anti-MuSK antibody mu07D09A targets NMJ, we conducted immunohistostaining on human muscle sections. Normal human skeletal muscle paraffin sections (TissueArray.Com, HuFPT075) were rehydrated. Heat antigen retrieval was performed by baking slides for 1 h at 60° C. Sections were permeabilized with 0.1% Triton-X₁₀₀ in 1× PBS for 15 min at RT and blocked in 5% horse serum in PBS for 30 min at RT. Sections were incubated with 10 μg/mL mu07D09A and 2 μg/mL Alexa Fluor™ 594 Alpha-bungarotoxin (Invitrogen, B13423) in incubation buffer (1% bovine serum albumin, 1% goat serum in PBS) overnight at RT. After 3 washes in PBS, sections were incubated with 10 μg/mL Goat anti-Mouse IgG, Alexa Fluor™ Plus 488 (Invitrogen, A32723) for 1 h at RT. Sections were sealed with VECTASHIELD® Antifade Mounting Medium with DAPI (Vector Laboratories, H-1200-10) and covered with coverslip. Sections were then imaged under a wide-field fluorescent microscope. Acetylcholine receptor (AChR) on human muscle sections was clearly labelled with alpha-bungarotoxin. mu07D09A co-localized well with AChR, suggesting that 07D09 binds NMJ on human muscle sections (FIG. 5).

To test whether mu07D09A-PRADshort and mu07D09A-PRADlong target NMJ, a similar IHC staining on human skeletal muscle sections was conducted. Both mu07D09A-PRADshort and mu07D09A-PRADlong co-localized with AChR, indicating NMJ binding of two APCs (FIG. 5).

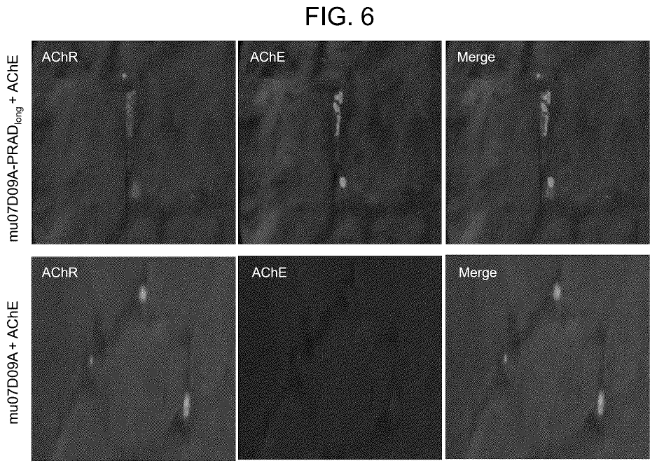

Mu07D09A-PRAD recruitment of AChE to NMJ on human skeletal muscle sections. To assess the ability of the anti-MuSK PRAD APC to anchor AChE at the NMJ and restore AChE presence at the NMJ, mu07D09A-PRADlong or mu07D09A were mixed with His-tagged recombinant AChE in incubation buffer (1% bovine serum albumin, 1% goat serum in PBS) for 1 h at 4° C. The mixture was then added to rehydrated normal human skeletal muscle sections and incubated overnight at 4° C. After 3 washes in PBS, the sections were incubated with 2 μg/mL Alexa Fluor™ 594 Alpha-bungarotoxin (Invitrogen, B13423) and 1 μg/mL His Tag Alexa Fluor®405-conjugated Antibody (R&D Systems, IC0501V) for 1 h at RT to reveal NMJ and His-AChE. After 3 washes with PBS, sections were sealed with VECTASHIELD® Antifade Mounting Medium (Vector Laboratories, H-1900-10) and covered with coverslip. The result showed that His-AChE was localized to the NMJ when mu07D09A-PRADlong was co-applied (FIG. 6). This is a surprising result as it suggests that anti-MuSK-PRAD APC can recruit exogenous AChE to the NMJ in wild-type mice, where endogenous CoIQ-tailed AChE is intact and already present. In fact, to the best of the inventors' knowledge, no synthetic construct has been reported to be, capable of recruiting AChE to the NMJ.

Conversely, His-AChE was not detected on the NMJ when mu07D09A was co-applied (FIG. 6). This result indicates that mu07D09-PRADlong effectively couples AChE to the NMJ. These data demonstrate that an anti-MuSK-PRAD APC can function as a surrogate for CoIQ and restore AChE presence at the NMJ.

Example 7. Generation of hu02C13A-PRAD APC and hu07D09A-PRAD APC. Traditional random or non-specific chemical conjugation methods have several drawbacks in the generation of antibody-peptide conjugates, including producing heterogeneous products and lacking reproducibility. Additionally, modifications near the complementarity-determining region (CDR) can negatively impact binding affinity and specificity. In contrast, site-specific conjugation techniques result in well-defined, more homogeneous antibody conjugates, improving both developability and clinical application potential, while achieving desired properties and functions.

Two popular site-specific approaches-chemo-enzymatic and chemical techniques-are commonly employed for antibody conjugation and are suitable for use in the generation of the anti-MuSK antibody conjugates described herein. One of chemo-enzymatic techniques involves glycan remodeling of the conserved N-glycan in the Fc region of immunoglobulin G (IgG). This approach was used to generate fully human anti-MuSK-PRAD APCs, i.e. hu02C13A-PRAD and hu07009A-PRAD.

02C13A and 07D09A were converted into fully human IgG1 with LALAPG mutations in the Fc region to abolish immune effector functions. The antibody-peptide conjugation process consists of two main steps. First, hu02C13 and hu07 009 were functionalized by introducing a biorthogonal DBCO group via Endo S2 which catalyzed site-specific glycan remodeling in a one-pot manner (deglycosylation and transglycosylation with the same enzyme), similar to the approaches developed by several groups. In the second step, the azide-activated PRADlong peptide was conjugated to the DBCO-functionalized antibodies using click chemistry via a cycloaddition reaction. Additional information on suitable methods to prepare the instant ADC and/or APC can be found at Zhang et al. (ACS Chem. Biol. (2021) 16(11): 2502-2514) and WO2024102523, which are hereby incorporated by reference in their entirety.

To generate a sufficient amount of homogeneous antibody-peptide conjugates, small-scale conjugation reactions (1-2 mg of antibodies) were first conducted for both antibodies to optimize reaction conditions (2 rounds, 2-3 reactions per round). Once optimal conditions were established, the process was scaled up, using 30 mg of each antibody (hu02C13A and hu07009A) under the optimized parameters.

BDCO-functionalized antibodies were generated as described previously and purified using a protein A column, and peptide conjugation was performed at a 1:5 peptide to antibody molar ratio at 4° C. for 16 hours. The final conjugate products were purified using Amicon columns (MWCO 30K) and formulated in PBS at pH 7.4. The analytical results and parameters for both antibody-peptide conjugates are summarized in Table 17. The peptide-to-antibody ratio (PAR) for both hu07D09A-PRAD and hu02C13A-PRAD was approximately 2 (2 PRAD peptides conjugated to each antibody).

TABLE 17
Analytical results of hu07D09A-PRAD and hu02C13A-PRAD.

| | | | | | | Free | |
|---|---|---|---|---|---|---|---|
| | UV Conc. | Amount | Yield | MS- | Monomer | peptide | Endotoxin |
| APC ID | (mg/mL) | (mg) | (%) | PAR* | (%) | (%) | (EU/mg) |
| 02C13- | 2.26 | 18.98 | 49.95 | 1.9 | 98.83 | <2.5 | <0.177 |
| peptide | | | | | | | |
| 07D09- | 2.42 | 22.26 | 58.58 | 1.86 | 98.38 | <2.5 | <0.165 |
| peptide | | | | | | | |

*UV chromatograph of reduced LC-MS was used to determine the PAR values

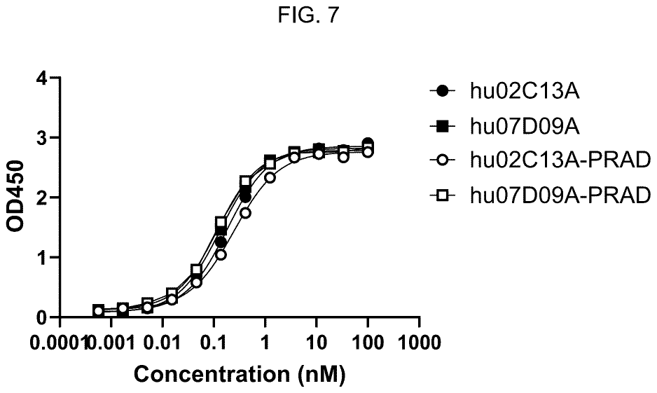

Example 8. In-vitro characterization of hu02C13A-PRAD and hu07D09A-PRAD. To test whether hu02C13A-PRAD and hu07D09A-PRAD could still bind MuSK, an ELISA assay against huMuSK ECD was conducted. High-binding 96-well plates (Corning, 9018) were coated with 100 μL per well of huMuSK ECD (2 μg/mL in PBS) overnight at 4° C. After 3 washes with PBST, each plate was blocked with 1% BSA in PBS for 1 h at room temperature (RT). Diluents of hu02C13A, hu07D09A, hu02C13A-PRAD and hu07D09A-PRAD (3-fold serial dilutions with concentrations in the range of 0-100 nM) were added to plates. The plate was incubated for 1 h at RT and washed 3 times with PBST. The plate was then incubated with HRP conjugated goat anti-mouse IgG (Thermo Fisher Scientific, G-21040) 1:5000 diluted in 1% BSA in PBS for 1 h at RT and washed 3 times. 3,3′, 5,5′-tetramethylbenzidine (TMB) Stabilized Chromogen substrate solution (Thermo Fisher Scientific, SB02) was added to the plate, and the reaction was stopped by adding 1M sulfuric acid. The absorbance at 450 nm was measured in a plate reader. The results of the ELISA assay are shown in FIG. 7. The EC50 of hu02C13A, hu07D09A, hu02C13A-PRAD and hu07D09A-PRAD were 0.18, 0.13, 0.25, and 0.12 nM, respectively. hu02C13A-PRAD and hu07D09A-PRAD have affinity with huMuSK ECD comparable with their corresponding parental antibodies.

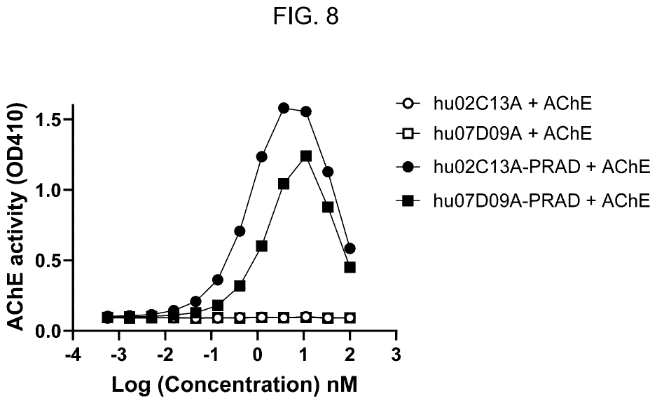

To test whether hu02C13A-PRAD and hu07D09A-PRAD could bind both MuSK and AChE, an AChE binding assay was conducted. Purified recombinant human AChE (6 μg/mL or 88 nM or 16.5 U/mL, made in house) was mixed with serial dilutions (3-fold serial dilutions with concentrations in the range of 0-15 μg/mL (0-100 nM)) of hu02C13A, hu07D09A, hu02C13A-PRAD, or hu07D09A-PRAD overnight at 4° C. In the meantime, a high-binding 96-well plate (Corning, 9018) was coated with 100 μL per well of huMuSK ECD (2 μg/mL in PBS) overnight at 4° C. After 3 washes with PBST, the plate was blocked with 1% BSA in PBS for 1 h at RT. The antibody-AChE mixture (100 μL per well) was added to the plate and incubated for 2 h on shaker at 4° C. After 3 washes with PBST, the AChE activity on the plate was measured by Acetylcholinesterase Assay Kit (Abcam, ab138871) according to manufacturer's instruction. The results of the AChE binding assay are in FIG. 8. AChE activity was detected on the plate when mixed with hu02C13A-PRAD or hu07D09A-PRAD but not with hu02C13A or hu07D09A alone, suggesting that hu02C13A-PRAD and hu07D09A-PRAD recruit AChE to MuSK. Surprisingly, AChE activity peaked when the concentration of hu02C13A-PRAD or hu07D09A-PRAD reached 11 nM and decreased when the concentration of the anti-MuSK-PRAD APC went higher. It suggests saturation of APC-AChE binding at 11 nM and competition from unbound APC at higher concentrations. When APC concentration went higher than 11 nM, there were unbound APCs in the mixture. The unbound APCs competed with APC-AChE complex for MuSK binding on the plate, causing a decrease of AChE detected on the plate. Under saturated binding, the concentrations of the APC and AChE in the mixture was 11 nM and 88 nM, respectively, corresponding to a 1:8 molar ratio of APC to AChE. This suggests that one anti-MuSK PRAD APC binds to eight AChE molecules. This ratio fits precisely with the PAR2 format of the APC (2 PRAD peptides per antibody) and the known pattern of PRAD with AChE (4 AChE molecules per PRAD peptide).

Example 9. Target engagement study of hu02C13A-PRAD and hu07D09A-PRAD in C57BL/6J mice. hu02C13A, hu07D09A, hu02C13A-PRAD, and hu07D09A-PRAD were injected i.p. at the dose of 10 mg/kg to adult C57BL/6J mice. Tibialis anterior (TA) muscles were isolated at 3, 7, and 28 days after the injection for IHC. After incubation in 2% PFA in 1×PBS for 2 hours at room temperature and in 30% sucrose in 1×PBS overnight at 4 degrees, the TA muscles were embedded in optimal cutting temperature (OCT) blocks and sectioned longitudinally at 20 μm onto SuperFrost slides (Fisher Scientific). Sections were immediately post-fixed and blocked. Triple IHC staining was then conducted on muscle sections. Bungarotoxin Alexa Fluor™ 647 (B35450, Invitrogen, 1:50), anti-acetylcholinesterase (AChE) antibody (ab183591, Abcam, 1:500), and goat anti-human IgG conjugated Alexa Fluor™ 488 (A-11013, Thermo Fisher, 1:200) was applied to label neuromuscular junction (NMJ), AChE, and injected human antibody, respectively. The secondary antibody for anti-AChE antibody was Goat anti-Rabbit IgG (H+L) secondary antibody, Alexa Fluor™ 546 (A-11035, Thermo Fisher). The whole slide images were generated using the Pannoramic SCAN (3D Histech) system. For image analysis, tissue areas on whole slide images were automatically detected. NMJs stained with Bungarotoxin were identified. Detected NMJs were further classified based on co-localization with anti-acetylcholinesterase and anti-human IgG markers. Morphological metrics were also calculated to assess NMJ shapes.

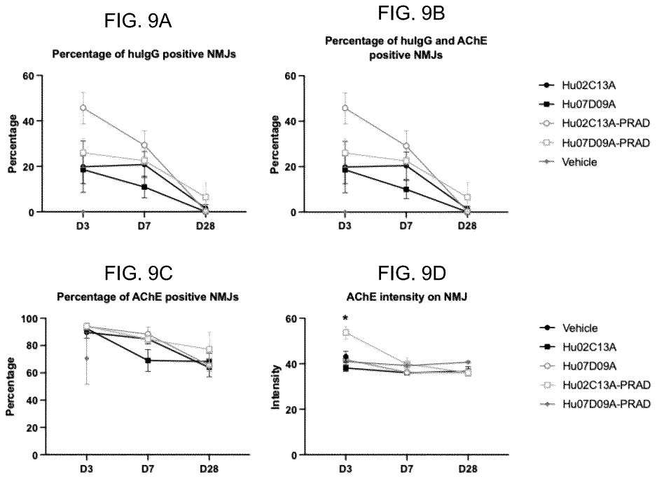

Results of image analysis are depicted in FIG. 9. Human IgG (huIgG) positive NMJs and huIgG and AChE positive NMJs were found in hu02C13A, hu07D09A, hu02C13A-PRAD, and hu07D09A-PRAD treated mice, but were not detected in vehicle-treated mice after injection (FIGS. 9A and B). These findings suggests that all four antibody preparations target NMJ in muscles after systemic injection. Around 50% of NMJ was hu02C13A-PRAD positive in mice at day 3 after hu02C13A-PRAD injection. Around 30% of NMJ was hu07D09A-PRAD positive at day 3. The proportion of huIgG positive NMJs was observed to decrease by day 7 and 28 after the injection. More than 60% of NMJs were AChE positive in all groups (FIG. 9C), which can be explained by the fact that AChE is located at NMJ in wild-type animals. Surprisingly, AChE intensity was significantly higher in hu02C13A-PRAD treated mice than that in vehicle-treated mice (FIG. 9D). This finding suggests that hu02C13A-PRAD recruited more endogenous AChE to NMJs in wild-type mice. This recruitment is unexpected as AChE is anchored to the NMJ via its binding with proteins on NMJ, such as CoIQ and MuSK. Those protein interactions were intact in WT mice, and thus, the AChE concentration in the NMJ was expected to be saturated in WT mice. Furthermore, it was surprising that the same effects of hu02C13A-PRAD were not observed for hu07D09A-PRAD.

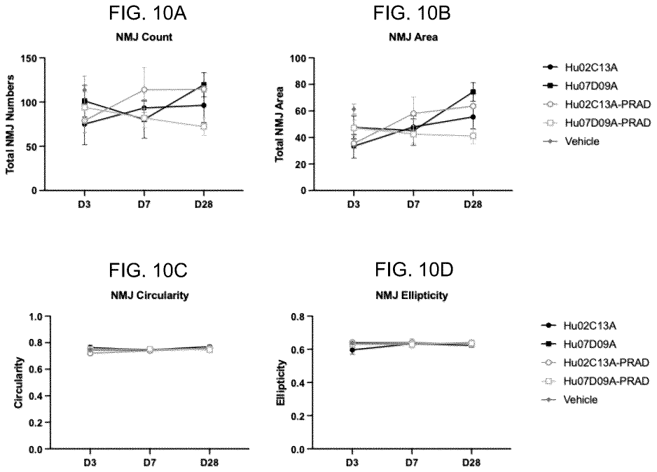

NMJ numbers and morphological metrics of the NMJ such as area, circularity, and ellipticity were not affected by any treatment (FIG. 10), which suggests that administration did not affect NMJ density or morphology.

Example 10. Generation of hu02C13A-PRAD APC with inter-cysteine conjugation. Another format of hu02C13A-PRAD APC, named as hu02C13A-PRAD (Cys), was generated by a chemical approach via cysteines using WuXiDAR2 technology developed by WuXi XDC for the creation of highly homogeneous antibody-drug conjugates (ADCs) with a specific drug-to-antibody ratio (DAR) of 2. The procedure involves selectively reducing one specific disulfide bond in the native antibody, yielding exactly two reactive thiol groups for conjugation with linker-payloads.

To generate a sufficient amount of homogeneous antibody-peptide conjugates, small-scale conjugation reactions (1-2 mg of antibodies) were first conducted for the antibody to optimize reaction conditions (2 rounds, 2-3 reactions per round). Once optimal conditions were established, the process was scaled up, using 20 mg of hu02C13A under the optimized parameters.

BDCO-functionalized antibody was generated by selectively reducing an interchain disulfide bond between two heavy chains of the antibody followed by a maleimide-cysteine reaction using a maleimide-PEG4-DBCO linker. Subsequently, an azide-activated PRADlong peptide was conjugated to the DBCO-functionalized antibody using click chemistry via a cycloaddition reaction. The resulting conjugate product was purified using AKTA-S200 and Amicon column (MWCO 30K). The formulation buffer is PBS, pH 7.4.

TABLE 6
Analytical results of hu02C13A-PRAD (Cys).

| | UV | | | | | Free | |
|---|---|---|---|---|---|---|---|
| | Conc. | Amount | Yield | MS- | Monomer | peptide | Endotoxin |
| APC ID | (mg/mL) | (mg) | (%) | PAR* | (%) | (%) | (EU/mg) |
| Hu02C13A- | 3.08 | 9.54 | 41.30 | 1.84 | 98.55 | <2 | 0.242 |
| PRAD | | | | | | | |
| (Cys) | | | | | | | |

*UV chromatograph of reduced LC-MS was used to determine the PAR values.

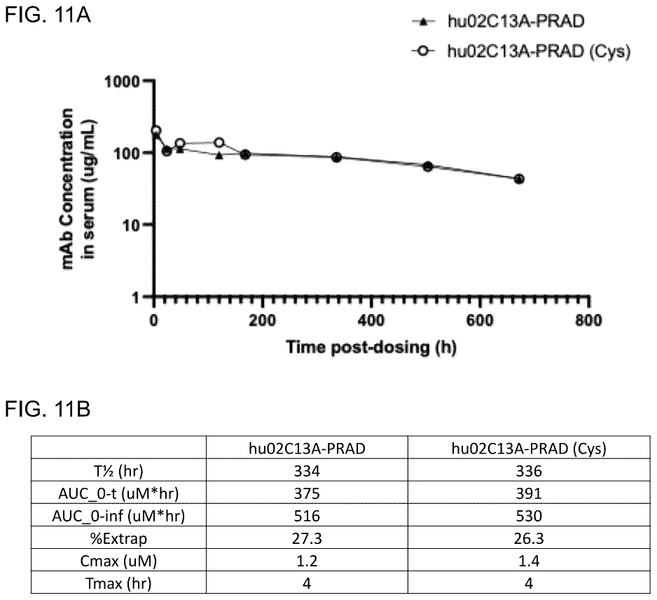

Example 11. Pharmacokinetic study of Hu02C13A-PRAD and hu02C13A-PRAD (Cys) in C57BL/6J mice. Hu02C13A-PRAD and hu02C13A-PRAD (Cys) were injected intraperitoneally (ip) in adult male C57BL/6JJ mice (The Jackson Laboratory, Strain #000664) at a dose of 10 mg/kg. Serum samples were collected at 0, 4 hours, 1, 2, 5, 7, 10, 14, 21, and 28 days after the injection (Serum samples from 3 mice each antibody per time point). Antibody levels in serum were measured by ELISA with human MuSK ECD as the coated antigen. High-binding 96-well plates (Corning, 9018) were coated with 100 μL per well of 2 μg/mL purified human MuSK extracellular doman (huMuSK ECD) overnight at 4° C. After 3 washes with PBST, each plate was blocked with 1% BSA in PBS for 1 h at RT. Serial dilutions of purified antibody (initial dilution 1.5 ng/mL, 3-fold dilutions, 8-points in duplicate) as standards or serum (initial dilution 1:500, 3-fold dilutions, 3-points) were added to plates. The plate was incubated for 1 h at RT and washed 3 times with PBST. The plate was then incubated with HRP conjugated goat anti-mouse IgG (Thermo Fisher Scientific, G-21040) 1:5000 diluted in 1% BSA in PBS for 1 h at RT and washed 3 times. TMB Stabilized Chromogen substrate solution (Thermo Fisher Scientific, SB02) was added to the plate, and the reaction was stopped by adding 1M sulfuric acid. The absorbance at 450 nm was measured in a plate reader. Non-compartmental PK analysis was conducted via winNonlin software (version 8.3). The PK profiles of antibodies are shown in FIG. 11. hu02C13-PRAD and hu02C13-PRAD-WuXiDAR2 had substantially similar PK profiles in C57BL/6J mice. The serum half-life of hu02C13-PRAD and hu02C13-PRAD (Cys) were 334 and 336 hours, respectively.

Example 12. Binding of Hu02C13A-PRAD and hu02C13A-PRAD (Cys) with endogenous AChE in mouse skeletal muscle. To test whether hu02C13A-PRAD and hu02C13A-PRAD (Cys) target endogenous AChE in skeletal muscle after systemic injection, hu02C13A-PRAD and hu02C13A-PRAD (Cys) were injected intraperitoneally (ip) in adult male C57BL/6J mice (The Jackson Laboratory, Strain #000664) at a dose of 10 mg/kg. To enhance muscle delivery of the PRAD peptide, sciatic nerve transection, which boosts MuSK protein expression in muscle, was conducted in the right hindlimb of the mice 2 days prior to the injection. Gastrocnemius (GC) muscle from both legs was isolated at 7 days after the injection. Muscle tissue was homogenized in PIERCE IP lysis buffer (Thermo Fisher Scientific, 87787) plus Halt Protease & Phosphatase inhibitor cocktail (Thermo Fisher Scientific, 78442) using a Bullet Blender (Next Advance). The homogenate was centrifuged at 21,000 g for 20 min at 4 degrees to remove insoluble fraction. Supernatant was mixed with 30 μL of anti-Human IgG Magnetic Beads (RayBiotech, 801-101) for 1 hour at 4 degrees to pull down Hu02C13A-PRAD and any binding proteins. The beads were washed 4 times with 500 μL of 0.05% Tween-20 in 1× TBS. 60 μL of elution buffer (0.1 M Glycine pH2.0) was added to the beads and beads were incubated for 10 min at RT. 52 μL of eluent was transferred to a new tube containing 8 μL of neutralization buffer (1M Tris pH8.0). 4× NuPAGE LDS sample buffer (Invitrogen, NP0007) with or without 10× NuPAGE reducing agent (Invitrogen, NP0004) was then added. Samples were incubated for 10 min at 70 degrees before proceeding to SDS-PAGE and Western Blot (WB) with anti-MuSK (R&D systems, AF562, 1:1000), anti-AChE (Invitrogen, MA5-42348, 1:1000), and anti-human IgG antibodies (Novus Bio, NB7489) to reveal MuSK, AChE, and Hu02C13A-PRAD.

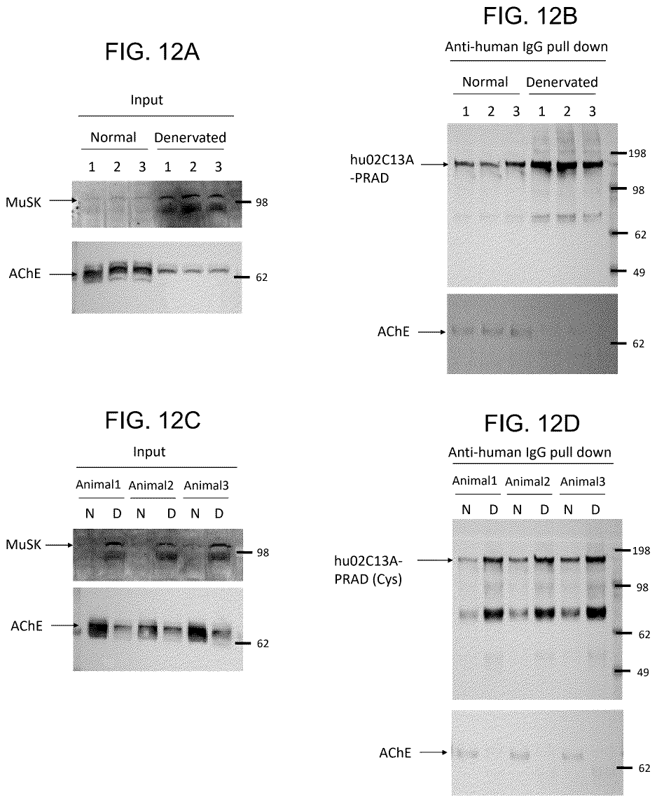

The WB results are provided in FIG. 12. FIGS. 12 A and B show the binding of hu02C13-PRAD and AChE in muscles collected 7 days after the injection. FIGS. 12 C and D show the binding of hu02C13-PRAD (Cys) and AChE in muscles collected 7 days after the injection. The WB results show that MuSK expression was elevated and AChE expression was reduced in muscle preparations isolated from mice that underwent nerve transection (FIGS. 12A and C). As expected, the concentration of the hu02C13-PRAD and hu02C13-PRAD (Cys) was higher in the denervated muscle as compared to the normal muscle (FIGS. 12B and D), suggesting that higher MuSK expression leads to greater PRAD distribution. AChE was co-pulled down with hu02C13A-PRAD and hu02C13-PRAD (Cys) in normal muscles (FIGS. 12B and D), suggesting that hu02C13A-PRAD binds endogenous AChE. There was no co-pulldown of AChE in the denervated muscle. Without being bound by a particular theory, this finding is likely because the AChE expression in the denervated muscle was too low.

### Embodiments

The present disclosure notes that various embodiments are disclosed herein, including:
A. An anti-MuSK antibody comprising:
   a variable heavy chain comprising:
      a complementarity determining region (CDR)1 with the amino acid sequence set forth in SEQ ID NOs: 3, 11, 19, 27, 35, 43, 51, 59, 67, 75, 83, or 91;
      a CDR2 with the amino acid sequence set forth in SEQ ID NOs: 4, 12, 20, 28, 36, 44, 52, 60, 68, 76, 84, or 92; and
      a CDR3 with the amino acid sequence set forth in SEQ ID NOs: 5, 13, 21, 29, 37, 45, 53, 61, 69, 77, 85, or 93; and
   a variable light chain comprising:
      a CDR1 with the amino acid sequence set forth in SEQ ID NOs: 7, 15, 23, 31, 39, 47, 55, 63, 71, 79, 87, or 95, 103, 111, 119, 127, 135, 143, 151, 159, 167, or 175;
      a CDR2 with the amino acid sequence set forth in SEQ ID NOs: 8, 16, 24, 32, 40, 48, 56, 64, 72, 80, 88, or 96; and
a CDR3 with the amino acid sequence set forth in SEQ ID NOs: 9, 17, 25, 33, 41, 49, 57, 65, 73, 81, 89, or 97.
   A1. The antibody of embodiment A, wherein the variable heavy chain of the anti-MuSK antibody comprises a CDR1 with the amino acid sequence set forth in SEQ ID NO:3, a CDR2 with the amino acid sequence set forth in SEQ ID NO:4, and a CDR3 with the amino acid sequence set forth in SEQ ID NO:5; and the variable light chain comprises of the anti-MuSK antibody a CDR1 with the amino acid sequence set forth in SEQ ID NO:7, a CDR2 with the amino acid sequence set forth in SEQ ID NO:8, and a CDR3 with the amino acid sequence set forth in SEQ ID NO:9.
      A1(i). The antibody of embodiment A or A1, wherein anti-MuSK antibody comprises one conservative substitution in SEQ ID NO: 3, 4, 5, 7, 8, and/or 9.
      A1(ii). The antibody of embodiment A or any preceding embodiment of A1 (i.e., A1 and A1(i)), wherein the variable heavy chain of the anti-MuSK antibody comprises a sequence with at least 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:2
      A1(iii). The antibody of embodiment A or any preceding embodiment of A1 (i.e., A1, A1(i), and A1(ii)), wherein the variable heavy chain of the anti-MuSK antibody comprises or consists of the amino acid sequence set forth in SEQ ID NO:2.
      A1(iv). The antibody of embodiment A or any preceding embodiment of A1 (i.e., A1, A1(i), A1(ii), or A1(iii)), wherein the variable light chain of the anti-MuSK antibody comprises a sequence with at least 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:6.
      A1(v). The antibody of embodiment A or any preceding embodiment of A1 (i.e., A1, A1(i), A1(ii), A1(iii), or A1(iv)), wherein the variable light chain of the anti-MuSK antibody comprises or consists of the amino acid sequence set forth in SEQ ID NO:6.
   A2. The antibody of embodiment A, wherein the variable heavy chain of the anti-MuSK antibody comprises a CDR1 with the amino acid sequence set forth in SEQ ID NO:11, a CDR2 with the amino acid sequence set forth in SEQ ID NO:12, and a CDR3 with the amino acid sequence set forth in SEQ ID NO:13; and the variable light chain comprises of the anti-MuSK antibody a CDR1 with the amino acid sequence set forth in SEQ ID NO:15, a CDR2 with the amino acid sequence set forth in SEQ ID NO:16, and a CDR3 with the amino acid sequence set forth in SEQ ID NO:17.
      A2(i). The antibody of embodiment A or A2, wherein the anti-MuSK antibody comprises one conservative substitution in SEQ ID NO: 11, 12, 13, 15, 16, and/or 17.
      A2(ii). The antibody of embodiment A or any preceding embodiment of A2, wherein the variable heavy chain of the anti-MuSK antibody comprises a sequence with at least 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:10.
      A2(iii). The antibody of embodiment A or any preceding embodiment of A2, wherein the variable heavy chain of the anti-MuSK antibody comprises or consists of the amino acid sequence set forth in SEQ ID NO:10.
      A2(iv). The antibody of embodiment A or any preceding embodiment of A2, wherein the variable light chain of the anti-MuSK antibody comprises a sequence with at least 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:14.
      A2(v). The antibody of embodiment A or any preceding embodiment of A2, wherein the variable light chain of the anti-MuSK antibody comprises or consists of the amino acid sequence set forth in SEQ ID NO:14.
   A3. The antibody of embodiment A, wherein the variable heavy chain of the anti-MuSK antibody comprises a CDR1 with the amino acid sequence set forth in SEQ ID NO:19, a CDR2 with the amino acid sequence set forth in SEQ ID NO:20, and a CDR3 with the amino acid sequence set forth in SEQ ID NO:21; and the variable light chain of the anti-MuSK antibody comprises a CDR1 with the amino acid sequence set forth in SEQ ID NO:23, a CDR2 with the amino acid sequence set forth in SEQ ID NO:24, and a CDR3 with the amino acid sequence set forth in SEQ ID NO:25.
      A3(i). The antibody of embodiment A or any preceding embodiment of A3, wherein the anti-MuSK antibody comprises one conservative substitution in SEQ ID NO: 19, 20, 21, 23, 24, and/or 25.
      A3(ii). The antibody of embodiment A or any preceding embodiment of A3, wherein the variable heavy chain of the anti-MuSK antibody comprises a sequence with at least 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:18
      A3(iii). The antibody of embodiment A or any preceding embodiment of A3, wherein the variable heavy chain of the anti-MuSK antibody comprises or consists of the amino acid sequence set forth in SEQ ID NO:18.
      A3(iv). The antibody of embodiment A or any preceding embodiment of A3, wherein the variable light chain of the anti-MuSK antibody comprises a sequence with at least 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:22.
      A3(v). The antibody of embodiment A or any preceding embodiment of A3, wherein the variable light chain of the anti-MuSK antibody comprises or consists of the amino acid sequence set forth in SEQ ID NO:22.
   A4. The antibody of embodiment A, wherein the variable heavy chain of the anti-MuSK antibody comprises a CDR1 with the amino acid sequence set forth in SEQ ID NO:27, a CDR2 with the amino acid sequence set forth in SEQ ID NO:28, and a CDR3 with the amino acid sequence set forth in SEQ ID NO:29; and the variable light chain of the anti-MuSK antibody comprises a CDR1 with the amino acid sequence set forth in SEQ ID NO:31, a CDR2 with the amino acid sequence set forth in SEQ ID NO:32, and a CDR3 with the amino acid sequence set forth in SEQ ID NO:33.
      A4(i). The antibody of embodiment A or A4, wherein the anti-MuSK antibody comprises one conservative substitution in SEQ ID NO: 27, 28, 29, 31, 32, and/or 33.
      A4(ii). The antibody of embodiment A or any preceding embodiment of A4, wherein the variable heavy chain of the anti-MuSK antibody comprises a sequence with at least 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:26
      A4(iii). The antibody of embodiment A or any preceding embodiment of A4, wherein the variable heavy chain of the anti-MuSK antibody comprises or consists of the amino acid sequence set forth in SEQ ID NO:26.
      A4(iv). The antibody of embodiment A or any preceding embodiment of A4, wherein the variable light chain of the anti-MuSK antibody comprises a sequence with at least 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:30.
      A4(v). The antibody of embodiment A or any preceding embodiment of A4, wherein the variable light chain of the anti-MuSK antibody comprises or consists of the amino acid sequence set forth in SEQ ID NO:30.
   A5. The antibody of embodiment A, wherein the variable heavy chain of the anti-MuSK antibody comprises a CDR1 with the amino acid sequence set forth in SEQ ID NO:35, a CDR2 with the amino acid sequence set forth in SEQ ID NO:36, and a CDR3 with the amino acid sequence set forth in SEQ ID NO:37; and the variable light chain of the anti-MuSK antibody comprises a CDR1 with the amino acid sequence set forth in SEQ ID NO:39, a CDR2 with the amino acid sequence set forth in SEQ ID NO:40, and a CDR3 with the amino acid sequence set forth in SEQ ID NO:41.
      A5(i). The antibody of embodiment A or A5, wherein the anti-MuSK antibody comprises one conservative substitution in SEQ ID NO: 35, 36, 37, 39, 40, and/or 41.
      A5(ii). The antibody of embodiment A or any preceding embodiment of A5, wherein the variable heavy chain of the anti-MuSK antibody comprises a sequence with at least 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:34
      A5(iii). The antibody of embodiment A or any preceding embodiment of A5, wherein the variable heavy chain of the anti-MuSK antibody comprises or consists of the amino acid sequence set forth in SEQ ID NO:34.
      A5(iv). The antibody of embodiment A or any preceding embodiment of A5, wherein the variable light chain of the anti-MuSK antibody comprises a sequence with at least 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:38.
      A5(v). The antibody of embodiment A or any preceding embodiment of A5, wherein the variable light chain of the anti-MuSK antibody comprises or consists of the amino acid sequence set forth in SEQ ID NO:38.
   A6. The antibody of embodiment A, wherein the variable heavy chain of the anti-MuSK antibody comprises a CDR1 with the amino acid sequence set forth in SEQ ID NO:43, a CDR2 with the amino acid sequence set forth in SEQ ID NO:44, and a CDR3 with the amino acid sequence set forth in SEQ ID NO:45; and the variable light chain of the anti-MuSK antibody comprises a CDR1 with the amino acid sequence set forth in SEQ ID NO:47, a CDR2 with the amino acid sequence set forth in SEQ ID NO:48, and a CDR3 with the amino acid sequence set forth in SEQ ID NO:49.
      A6(i). The antibody of embodiment A or A6, wherein the anti-MuSK antibody comprises one conservative substitution in SEQ ID NO: 43, 44, 45, 47, 48, and/or 49.
      A6(ii). The antibody of embodiment A or any preceding embodiment of A6, wherein the variable heavy chain of the anti-MuSK antibody comprises a sequence with at least 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:42
      A6(iii). The antibody of embodiment A or any preceding embodiment of A6, wherein the variable heavy chain of the anti-MuSK antibody comprises or consists of the amino acid sequence set forth in SEQ ID NO:42.
      A6(iv). The antibody of embodiment A or any preceding embodiment of A6, wherein the variable light chain of the anti-MuSK antibody comprises a sequence with at least 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:46.
      A6(v). The antibody of embodiment A or any preceding embodiment of A6, wherein the variable light chain of the anti-MuSK antibody comprises or consists of the amino acid sequence set forth in SEQ ID NO:46.
   A7. The antibody of embodiment A, wherein the variable heavy chain of the anti-MuSK antibody comprises a CDR1 with the amino acid sequence set forth in SEQ ID NO:51, a CDR2 with the amino acid sequence set forth in SEQ ID NO:52, and a CDR3 with the amino acid sequence set forth in SEQ ID NO:53; and the variable light chain of the anti-MuSK antibody comprises a CDR1 with the amino acid sequence set forth in SEQ ID NO:55, a CDR2 with the amino acid sequence set forth in SEQ ID NO:56, and a CDR3 with the amino acid sequence set forth in SEQ ID NO:57.
      A7(i). The antibody of embodiment A or A7, wherein the anti-MuSK antibody comprises one conservative substitution in SEQ ID NO: 51, 52, 53, 55, 56, and/or 57.
      A7(ii). The antibody of embodiment A or any preceding embodiment of A7, wherein the variable heavy chain of the anti-MuSK antibody comprises a sequence with at least 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:50
      A7(iii). The antibody of embodiment A or any preceding embodiment of A7, wherein the variable heavy chain of the anti-MuSK antibody comprises or consists of the amino acid sequence set forth in SEQ ID NO:50.
      A7(iv). The antibody of embodiment B, C, or any preceding embodiment of A7, wherein the variable light chain of the anti-MuSK antibody comprises a sequence with at least 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:54.
      A7(v). The antibody of embodiment A or any preceding embodiment of A7, wherein the variable light chain of the anti-MuSK antibody comprises or consists of the amino acid sequence set forth in SEQ ID NO:54.
   A8. The antibody of embodiment A, wherein the variable heavy chain of the anti-MuSK antibody comprises a CDR1 with the amino acid sequence set forth in SEQ ID NO:59, a CDR2 with the amino acid sequence set forth in SEQ ID NO:60, and a CDR3 with the amino acid sequence set forth in SEQ ID NO:61; and the variable light chain of the anti-MuSK antibody comprises a CDR1 with the amino acid sequence set forth in SEQ ID NO:63, a CDR2 with the amino acid sequence set forth in SEQ ID NO:64, and a CDR3 with the amino acid sequence set forth in SEQ ID NO:65.
      A8(i). The antibody of embodiment A or A8, wherein the anti-MuSK antibody comprises one conservative substitution in SEQ ID NO: 59, 60, 61, 63, 64, and/or 65.
      A8(ii). The antibody of embodiment A or any preceding embodiment of A8, wherein the variable heavy chain of the anti-MuSK antibody comprises a sequence with at least 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:58
      A8(iii). The antibody of embodiment A or any preceding embodiment of A8, wherein the variable heavy chain of the anti-MuSK antibody comprises or consists of the amino acid sequence set forth in SEQ ID NO:58.
      A8(iv). The antibody of embodiment A or any preceding embodiment of A8, wherein the variable light chain of the anti-MuSK antibody comprises a sequence with at least 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:62.
      A8(v). The antibody of embodiment A or any preceding embodiment of A8, wherein the variable light chain of the anti-MuSK antibody comprises or consists of the amino acid sequence set forth in SEQ ID NO:62.
   A9. The antibody of embodiment A, wherein the variable heavy chain of the anti-MuSK antibody comprises a CDR1 with the amino acid sequence set forth in SEQ ID NO:67, a CDR2 with the amino acid sequence set forth in SEQ ID NO:68, and a CDR3 with the amino acid sequence set forth in SEQ ID NO:69; and the variable light chain of the anti-MuSK antibody comprises a CDR1 with the amino acid sequence set forth in SEQ ID NO:71, a CDR2 with the amino acid sequence set forth in SEQ ID NO:72, and a CDR3 with the amino acid sequence set forth in SEQ ID NO:73.
      A9(i). The antibody of embodiment A or A9, wherein the anti-MuSK antibody comprises one conservative substitution in SEQ ID NO: 67, 68, 69, 71, 72, and/or 73.
      A(ii). The antibody of embodiment A or any preceding embodiment of A9, wherein the variable heavy chain of the anti-MuSK antibody comprises a sequence with at least 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:66
      A9(iii). The antibody of embodiment A or any preceding embodiment of A9, wherein the variable heavy chain of the anti-MuSK antibody comprises or consists of the amino acid sequence set forth in SEQ ID NO:66.
      A9(iv). The antibody of embodiment A or any preceding embodiment of A9, wherein the variable light chain of the anti-MuSK antibody comprises a sequence with at least 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:70.
      A9(v). The antibody peptide conjugate of embodiment A or any preceding embodiment of A9, wherein the variable light chain of the anti-MuSK antibody comprises or consists of the amino acid sequence set forth in SEQ ID NO:70.
   A10. The antibody of embodiment A, wherein the variable heavy chain of the anti-MuSK antibody comprises a CDR1 with the amino acid sequence set forth in SEQ ID NO:75, a CDR2 with the amino acid sequence set forth in SEQ ID NO:76, and a CDR3 with the amino acid sequence set forth in SEQ ID NO:77; and the variable light chain of the anti-MuSK antibody comprises a CDR1 with the amino acid sequence set forth in SEQ ID NO:79, a CDR2 with the amino acid sequence set forth in SEQ ID NO:80, and a CDR3 with the amino acid sequence set forth in SEQ ID NO:81.
      A10(i). The antibody of embodiment A or A10, wherein the anti-MuSK antibody comprises one conservative substitution in SEQ ID NO: 75, 76, 77, 79, 80, and/or 81.
      A10(ii). The antibody of embodiment A or any preceding embodiment of A10, wherein the variable heavy chain of the anti-MuSK antibody comprises a sequence with at least 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:74
      A10(iii). The antibody of embodiment A or any preceding embodiment of A10, wherein the variable heavy chain of the anti-MuSK antibody comprises or consists of the amino acid sequence set forth in SEQ ID NO:74.
      A10(iv). The antibody of embodiment A or any preceding embodiment of A10, wherein the variable light chain of the anti-MuSK antibody comprises a sequence with at least 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:78
      A10(v). The antibody of embodiment A or any preceding embodiment of A10, wherein the variable light chain of the anti-MuSK antibody comprises or consists of the amino acid sequence set forth in SEQ ID NO:78.
   A11. The antibody of embodiment A, wherein the variable heavy chain of the anti-MuSK antibody comprises a CDR1 with the amino acid sequence set forth in SEQ ID NO:83, a CDR2 with the amino acid sequence set forth in SEQ ID NO:84, and a CDR3 with the amino acid sequence set forth in SEQ ID NO:85; and the variable light chain of the anti-MuSK antibody comprises a CDR1 with the amino acid sequence set forth in SEQ ID NO:87, a CDR2 with the amino acid sequence set forth in SEQ ID NO:88, and a CDR3 with the amino acid sequence set forth in SEQ ID NO:89.
      A11(i). The antibody of embodiment A or A11, wherein the anti-MuSK antibody comprises one conservative substitution in SEQ ID NO: 83, 84, 85, 87, 88, and/or 89.
      A11(ii). The antibody of embodiment A or any preceding embodiment of A11, wherein the variable heavy chain of the anti-MuSK antibody comprises a sequence with at least 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:82
      A11(iii). The antibody of embodiment A or any preceding embodiment of A11, wherein the variable heavy chain of the anti-MuSK antibody comprises or consists of the amino acid sequence set forth in SEQ ID NO:82.
      A11(iv). The antibody of embodiment A or any preceding embodiment of A11, wherein the variable light chain of the anti-MuSK antibody comprises a sequence with at least 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:86
      A11(v). The antibody of embodiment A or any preceding embodiment of A11, wherein the variable light chain of the anti-MuSK antibody comprises or consists of the amino acid sequence set forth in SEQ ID NO:86.
   A12. The antibody of embodiment A, wherein the variable heavy chain of the anti-MuSK antibody comprises a CDR1 with the amino acid sequence set forth in SEQ ID NO:91, a CDR2 with the amino acid sequence set forth in SEQ ID NO:92, and a CDR3 with the amino acid sequence set forth in SEQ ID NO:93; and the variable light chain of the anti-MuSK antibody comprises a CDR1 with the amino acid sequence set forth in SEQ ID NO:95, a CDR2 with the amino acid sequence set forth in SEQ ID NO:96, and a CDR3 with the amino acid sequence set forth in SEQ ID NO:97.
      A12(i). The antibody of embodiment A or A12, wherein the anti-MuSK antibody one conservative substitution is present in SEQ ID NO: 91, 92, 93, 95, 96, and/or 97.
      A12(ii). The antibody of embodiment A or any preceding embodiment of A12, wherein the variable heavy chain of the anti-MuSK antibody comprises a sequence with at least 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:90
      A12(iii). The antibody of embodiment A or any preceding embodiment of A12, wherein the variable heavy chain of the anti-MuSK antibody comprises or consists of the amino acid sequence set forth in SEQ ID NO:90.
      A12(iv). The antibody of embodiment A or any preceding embodiment of A12, wherein the variable light chain of the anti-MuSK antibody comprises a sequence with at least 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO:94
      A12(v). The antibody of embodiment A or any preceding embodiment of A12, wherein the variable light chain of the anti-MuSK antibody comprises or consists of the amino acid sequence set forth in SEQ ID NO:94.
   A13. The antibody of any preceding embodiment, wherein the anti-MuSK antibody is a monoclonal antibody.
      A13(i). The antibody peptide conjugate of embodiment A13, wherein the monoclonal antibody is human or fully human.
      A13(ii). The anti-MuSK antibody of embodiment A13, wherein the monoclonal antibody is humanized.
   A14. The antibody of any preceding embodiment, wherein the anti-MuSK antibody is radiolabeled.
   A14. The antibody of embodiment A14, wherein the radiolabel is selected from the group consisting of: ¹³¹I, ⁹⁰Y ²¹²Bi, ¹⁸⁶Re, ²²¹At, 99mTc; or a mixture thereof.
   A15. The antibody of any preceding embodiment, wherein the antibody functions as a drug delivery vehicle.
B. A means for binding MuSK comprising a protein configured to bind MuSK and elicit a negligible effect on MuSK function.
C. The antibody or binding means of any preceding embodiment, wherein the anti-MuSK antibody or binding means is capable of binding MuSK (SEQ ID NO:1) or a variant thereof.
   C1. The antibody or means for binding MuSK of embodiment C, wherein the antibody or binding means binds to an epitope of the Fz domain of human MuSK.
   C2. The antibody or means for binding MuSK of embodiment C, wherein the antibody or binding means binds to an epitope of the Ig-3 domain of human MuSK.
   C3. The antibody or means for binding MuSK of any preceding embodiment of C, wherein the anti-MuSK antibody or binding means, when bound to MuSK, does not inhibit the function of MuSK.
   C4. The antibody or means for binding MuSK of any preceding embodiment, wherein the anti-MuSK antibody or binding means, when bound to MuSK, does not decrease MuSK function by more than 50%.
   C5. The antibody or means for binding MuSK of any preceding embodiment, wherein the anti-MuSK antibody or binding means, when bound to MuSK, does not decrease MuSK function by more than 25%.
   C6. The antibody or means for binding MuSK of any preceding embodiment, wherein the anti-MuSK antibody or binding means, when bound to MuSK, does not decrease MuSK function by more than 15%.
   C7. The antibody or means for binding MuSK of any preceding embodiment, wherein the anti-MuSK antibody or binding means, when bound to MuSK, does not decrease MuSK function by more than 10%.
   C8. The antibody or means for binding MuSK of any preceding embodiment, wherein the anti-MuSK antibody, when bound to MuSK, does not activate MuSK.
   C9. The antibody or means for binding MuSK of any preceding embodiment, when bound to MuSK, does not increase MuSK function by more than 50%.
   C10. The antibody or means for binding MuSK of any preceding embodiment, when bound to MuSK, does not increase MuSK function by more than 25%.
   C11. The antibody or means for binding MuSK of any preceding embodiment, wherein the anti-MuSK antibody, when bound to MuSK, does not increase MuSK function by more than 15%.
   C12. The antibody or means for binding MuSK of any preceding embodiment, wherein the anti-MuSK antibody, when bound to MuSK, does not increase MuSK function by more than 10%.
D. A proline-rich attachment domain (PRAD) peptide comprising a core sequence selected from the group consisting of SEQ ID NOs: 106, 107, and 108, and optionally comprising or consisting of a first sequence and/or a second sequence.
   D1. The PRAD peptide of embodiment D, further comprising or consisting of a first sequence located at the N-terminus of the core sequence.
      D1(i). The PRAD peptide of any preceding embodiment of D (i.e., D and D1), wherein the first sequence comprises or consists of 1, 2, 3, 4, 5, or 6 amino acids.
      D1(ii). The PRAD peptide of any preceding embodiment of D, wherein the amino acids of the first sequence are natural human amino acids.
   D2. The PRAD peptide of any preceding embodiment of D (i.e., D, D1, D1(i) and D1(ii)), further comprising a second sequence located at the C-terminus of the core sequence.
      D2(i). The PRAD peptide conjugate of any preceding embodiment of D, wherein the second sequence comprises 1, 2, 3, 4, 5, 6, 7, or 8 amino acids.
      D2(ii). The PRAD peptide of any preceding embodiment of D, wherein the amino acids of the second sequence are natural human amino acids.
   D3. The PRAD peptide of any preceding embodiment of D, wherein the PRAD peptide comprises or consists of 15, 16, 17, 18, 19, 20, 21, 22, 23, 24, 25, 26, 27, 28, or 29 amino acids.
   D4. The PRAD peptide of any preceding embodiment of D, wherein the first sequence comprises or consists of 1 amino acid, and the second sequence comprises or consists of 1, 2, 3, 4, 5, 6, 7, or 8 amino acids.
   D5. The PRAD peptide of any preceding embodiment of D, wherein the first sequence comprises or consists of 2 amino acids, and a second sequence comprises or consists of 1, 2, 3, 4, 5, 6, 7, or 8 amino acids.
   D6. The PRAD peptide of any preceding embodiment of D, wherein the first sequence comprises or consists of 3 amino acids, and a second sequence comprises or consists of 1, 2, 3, 4, 5, 6, 7, or 8 amino acids.
   D7. The PRAD peptide of any preceding embodiment of D, wherein the first sequence comprises or consists of 4 amino acids, and a second sequence comprises or consists of 1, 2, 3, 4, 5, 6, 7, or 8 amino acids.
   D8. The PRAD peptide of any preceding embodiment of D, wherein the first sequence comprises or consists of 5 amino acids, and a second sequence comprises or consists of 1, 2, 3, 4, 5, 6, 7, or 8 amino acids.
   D9. The PRAD peptide of any preceding embodiment of D, wherein the first sequence comprises or consists of 6 amino acids, and a second sequence comprises or consists of 1, 2, 3, 4, 5, 6, 7, or 8 amino acids.
   D10. The PRAD peptide of any preceding embodiment of D, wherein the second sequence comprises or consists of 1 amino acid, and a first sequence comprises or consists of 1, 2, 3, 4, 5, or 6 amino acids.
   D11. The PRAD peptide of any preceding embodiment of D, wherein the second sequence comprises or consists of 2 amino acids, and a first sequence comprises or consists of 1, 2, 3, 4, 5, or 6 amino acids.
   D12. The PRAD peptide of any preceding embodiment of D, wherein the second sequence comprises or consists of 3 amino acids, and a first sequence comprises or consists of 1, 2, 3, 4, 5, or 6 amino acids.
   D13. The PRAD peptide of any preceding embodiment of D, wherein the second sequence comprises or consists of 4 amino acids, and a first sequence comprises or consists of 1, 2, 3, 4, 5, or 6 amino acids.
   D14. The PRAD peptide of any preceding embodiment of D, wherein the second sequence comprises or consists of 5 amino acids, and a first sequence comprises or consists of 1, 2, 3, 4, 5, or 6 amino acids.
   D15. The PRAD peptide of any preceding embodiment of D, wherein the second sequence comprises or consists of 6 amino acids, and a first sequence comprises or consists of 1, 2, 3, 4, 5, or 6 amino acids.
   D16. The PRAD peptide of any preceding embodiment of D, wherein the second sequence comprises or consists of 7 amino acids, and a first sequence comprises or consists of 1, 2, 3, 4, 5, or 6 amino acids.
   D17. The PRAD peptide of any preceding embodiment of D, wherein the second sequence comprises or consists of 8 amino acids, and a first sequence comprises or consists of 1, 2, 3, 4, 5, or 6 amino acids.
   D18. The PRAD peptide of any embodiment of D to D3 (i.e. D, D1, D1(i), D1(ii), D2, D2(i), D2(ii), and D3), wherein the first sequence with 1 amino acid.
   D19. The PRAD peptide of any embodiment of D to D3, wherein the first sequence comprises or consists of 2 amino acids.
   D20. The PRAD peptide of any embodiment of D to D3, wherein the first sequence comprises or consists of 3 amino acids.
   D21. The PRAD peptide of any embodiment of D to D3, wherein the first sequence comprises or consists of 4 amino acids.
   D22. The PRAD peptide of any embodiment of D to D3, wherein the first sequence comprises or consists of 5 amino acids.
   D23. The PRAD peptide of any embodiment of D to D3, wherein the first sequence comprises or consists of 6 amino acids.
   D24. The PRAD peptide of any embodiment of D to D3 or D18 to D23, wherein the second sequence comprises or consists of 1 amino acid,
   D25. The PRAD peptide of any embodiment of D to D3 or D18 to D23, wherein the second sequence comprises or consists of 2 amino acids,
   D26. The PRAD peptide of any embodiment of D to D3 or D18 to D23, wherein the second sequence comprises or consists of 3 amino acids.
   D27. The PRAD peptide of any embodiment of D to D3 or D18 to D23, wherein the second sequence comprises or consists of 4 amino acids.
   D28. The PRAD peptide of any embodiment of D to D3 or D18 to D23, wherein the second sequence comprises or consists of 5 amino acids.
   D29. The PRAD peptide of any embodiment of D to D3 or D18 to D23, wherein the second sequence comprises or consists of 6 amino acids.
   D30. The PRAD peptide of any embodiment of D to D3 or D18 to D23, wherein the second sequence comprises or consists of 7 amino acids.
   D31. The PRAD peptide of any embodiment of D to D3 or D18 to D23, wherein the second sequence comprises or consists of 8 amino acids.
E. A PRAD peptide comprising a sequence according to formula (1): X₁-X₂-X₃-X₄-X₅-X₆-CORE-X₇-X₈-X-X₁₀-X₁₁-X₁₂-X₁₃-X₁₄, wherein X₁ is absent or any natural human amino acid, preferably C; X₂ is absent or any natural human amino acid, preferably C; X₃ is absent or any natural human amino acid; X₄ is absent or any natural human amino acid; X₅ is absent or any natural human amino acid; X₆ is absent or any natural human amino acid; X₇ is absent or any natural human amino acid, preferably R, G, or S; X₃ is absent or any natural human amino acid, preferably R, G, or S; X₉ is absent or any natural human amino acid, preferably R, G, or S; X₁₀ is absent or any natural human amino acid, preferably R, G, or S; X₁₁ is absent or any natural human amino acid, preferably R, G, or S; X₁₂ is absent or any natural human amino acid, preferably R, G, or S; X₁₃ is absent or any natural human amino acid; X₁₄ is absent or any natural human amino acid, and CORE is selected from the group of SEQ ID NO. 106, 107 and 108, preferably SEQ ID NO. 106.
F. An antibody peptide conjugate comprising, consisting or, or consisting essentially of: a means for binding MuSK according to any one of preceding embodiments B or C, or an anti-MuSK antibody according to any one of preceding embodiments A or C; and a PRAD peptide according to any one of the preceding embodiments of D or E.
G. An antibody-drug conjugate comprising, consisting or, or consisting essentially of: a means for binding MuSK according to any one of preceding embodiments B or C, or an anti-MuSK antibody according to any one of preceding embodiments A or C; and a drug.
   G1. The antibody-drug conjugate of embodiment G, further comprising, consisting or, or consisting essentially of: a PRAD peptide according to any one of the preceding embodiments of D or E.
   G2. The antibody-drug conjugate of any preceding embodiment of G (i.e., G and G1), wherein the drug is biologically active in the NMJ.
   G3. The antibody-drug conjugate of any preceding embodiment of G, wherein the drug comprises or is an acetylcholinesterase inhibitor.
   G4. The antibody-drug conjugate of any preceding embodiment of G, wherein the drug comprises or is an acetylcholine receptor inhibitor.
   G5. The antibody-drug conjugate of any preceding embodiment of G, wherein the drug comprises or is an adrenergic agonist.

What is claimed is:

1. An antibody peptide conjugate comprising a means for binding MuSK or an anti-MuSK antibody and a PRAD peptide.

2. The antibody peptide conjugate of claim 1, wherein the anti-MuSK antibody comprises:
a variable heavy chain comprising:
a complementarity determining region (CDR)1 with the amino acid sequence set forth in SEQ ID NOs:3, 11, 19, 27, 35, 43, 51, 59, 67, 75, 83, 91, 99, 107, 115, 123, 131, 139, 147, 155, 163, or 171;
a CDR2 with the amino acid sequence set forth in SEQ ID NOs: 4, 12, 20, 28, 36, 44, 52, 60, 68, 76, 84, 92, 100, 108, 116, 124, 132, 140, 148, 156, 164, or 172; and
a CDR3 with the amino acid sequence set forth in SEQ ID NOs: 5, 13, 21, 29, 37, 45, 53, 61, 69, 77, 85, 93, 101, 109, 117, 125, 133, 141, 149, 157, 165, or 173; and
a variable light chain comprising:
a CDR1 with the amino acid sequence set forth in SEQ ID NOs: 7, 15, 23, 31, 39, 47, 55, 63, 71, 79, 87, 95, 103, 111, 119, 127, 135, 143, 151, 159, 167, or 175;
a CDR2 with the amino acid sequence set forth in SEQ ID NOs: 8, 16, 24, 32, 40, 48, 56, 64, 72, 80, 88, 96, 104, 112, 120, 128, 136, 144, 152, 160, 168, or 176; and
a CDR3 with the amino acid sequence set forth in SEQ ID NOs: 9, 17, 25, 33, 41, 49, 57, 65, 73, 81, 89, 97, 105, 113, 121, 129, 137, 145, 153, 161, 169, or 177.

3. The antibody peptide conjugate of claim 2, wherein the PRAD peptide comprises a core sequence selected from the group consisting of SEQ ID NOs: 106, 107, and 108.

4. The antibody peptide conjugate of claim 3, wherein the PRAD peptide further comprises a first and/or a second sequence, wherein the first sequence is located at the N-terminus of the core sequence and the second sequence is located at the C-terminus of the core sequence.

5. The antibody peptide conjugate of claim 4, wherein the first sequence consists of 1 to 6 amino acids.

6. The antibody peptide conjugate of claim 5, wherein the second sequence consists of 1 to 8 amino acids.

7. An anti-MuSK antibody comprising:
a variable heavy chain comprising:
a complementarity determining region (CDR)1 with the amino acid sequence set forth in SEQ ID NOs:3, 11, 19, 27, 35, 43, 51, 59, 67, 75, 83, 91, 99, 107, 115, 123, 131, 139, 147, 155, 163, or 171;
a CDR2 with the amino acid sequence set forth in SEQ ID NOs: 4, 12, 20, 28, 36, 44, 52, 60, 68, 76, 84, 92, 100, 108, 116, 124, 132, 140, 148, 156, 164, or 172; and
a CDR3 with the amino acid sequence set forth in SEQ ID NOs: 5, 13, 21, 29, 37, 45, 53, 61, 69, 77, 85, 93, 101, 109, 117, 125, 133, 141, 149, 157, 165, or 173; and
a variable light chain comprising:
a CDR1 with the amino acid sequence set forth in SEQ ID NOs: 7, 15, 23, 31, 39, 47, 55, 63, 71, 79, 87, 95, 103, 111, 119, 127, 135, 143, 151, 159, 167, or 175;
a CDR2 with the amino acid sequence set forth in SEQ ID NOs: 8, 16, 24, 32, 40, 48, 56, 64, 72, 80, 88, 96, 104, 112, 120, 128, 136, 144, 152, 160, 168, or 176; and
a CDR3 with the amino acid sequence set forth in SEQ ID NOs: 9, 17, 25, 33, 41, 49, 57, 65, 73, 81, 89, 97, 105, 113, 121, 129, 137, 145, 153, 161, 169, or 177.

8. The anti-MuSK antibody of claim 7, wherein the variable heavy chain of the anti-MuSK antibody comprises a CDR1 with the amino acid sequence set forth in SEQ ID NO:11, a CDR2 with the amino acid sequence set forth in SEQ ID NO:12, and a CDR3 with the amino acid sequence set forth in SEQ ID NO:13; and the variable light chain comprises of the anti-MuSK antibody a CDR1 with the amino acid sequence set forth in SEQ ID NO:15, a CDR2 with the amino acid sequence set forth in SEQ ID NO:16, and a CDR3 with the amino acid sequence set forth in SEQ ID NO:17.

9. The anti-MuSK antibody of claim 7, wherein the variable heavy chain of the anti-MuSK antibody comprises a CDR1 with the amino acid sequence set forth in SEQ ID NO:19, a CDR2 with the amino acid sequence set forth in SEQ ID NO:20, and a CDR3 with the amino acid sequence set forth in SEQ ID NO:21; and the variable light chain of the anti-MuSK antibody comprises a CDR1 with the amino acid sequence set forth in SEQ ID NO:23, a CDR2 with the amino acid sequence set forth in SEQ ID NO:24, and a CDR3 with the amino acid sequence set forth in SEQ ID NO:25.

10. The anti-MuSK antibody of claim 7, wherein the variable heavy chain of the anti-MuSK antibody comprises a CDR1 with the amino acid sequence set forth in SEQ ID NO:27, a CDR2 with the amino acid sequence set forth in SEQ ID NO:28, and a CDR3 with the amino acid sequence set forth in SEQ ID NO:29; and the variable light chain of the anti-MuSK antibody comprises a CDR1 with the amino acid sequence set forth in SEQ ID NO:31, a CDR2 with the amino acid sequence set forth in SEQ ID NO:32, and a CDR3 with the amino acid sequence set forth in SEQ ID NO:33.

11. The anti-MuSK antibody of claim 7, wherein the variable heavy chain of the anti-MuSK antibody comprises a CDR1 with the amino acid sequence set forth in SEQ ID NO:35, a CDR2 with the amino acid sequence set forth in SEQ ID NO:36, and a CDR3 with the amino acid sequence set forth in SEQ ID NO:37; and the variable light chain of the anti-MuSK antibody comprises a CDR1 with the amino acid sequence set forth in SEQ ID NO:39, a CDR2 with the amino acid sequence set forth in SEQ ID NO:40, and a CDR3 with the amino acid sequence set forth in SEQ ID NO:41.

12. The anti-MuSK antibody of claim 7, wherein the variable heavy chain of the anti-MuSK antibody comprises a CDR1 with the amino acid sequence set forth in SEQ ID NO:43, a CDR2 with the amino acid sequence set forth in SEQ ID NO:44, and a CDR3 with the amino acid sequence set forth in SEQ ID NO:45; and the variable light chain of the anti-MuSK antibody comprises a CDR1 with the amino acid sequence set forth in SEQ ID NO:47, a CDR2 with the amino acid sequence set forth in SEQ ID NO:48, and a CDR3 with the amino acid sequence set forth in SEQ ID NO:49.

13. The anti-MuSK antibody of claim 7, wherein the variable heavy chain of the anti-MuSK antibody comprises a CDR1 with the amino acid sequence set forth in SEQ ID NO:51, a CDR2 with the amino acid sequence set forth in SEQ ID NO:52, and a CDR3 with the amino acid sequence set forth in SEQ ID NO:53; and the variable light chain of the anti-MuSK antibody comprises a CDR1 with the amino acid sequence set forth in SEQ ID NO:55, a CDR2 with the amino acid sequence set forth in SEQ ID NO:56, and a CDR3 with the amino acid sequence set forth in SEQ ID NO:57.

14. The anti-MuSK antibody of claim 7, wherein the variable heavy chain of the anti-MuSK antibody comprises a CDR1 with the amino acid sequence set forth in SEQ ID NO:59, a CDR2 with the amino acid sequence set forth in SEQ ID NO:60, and a CDR3 with the amino acid sequence set forth in SEQ ID NO:61; and the variable light chain of the anti-MuSK antibody comprises a CDR1 with the amino acid sequence set forth in SEQ ID NO:63, a CDR2 with the amino acid sequence set forth in SEQ ID NO:64, and a CDR3 with the amino acid sequence set forth in SEQ ID NO:65.

15. The anti-MuSK antibody of claim 7, wherein the variable heavy chain of the anti-MuSK antibody comprises a CDR1 with the amino acid sequence set forth in SEQ ID NO:67, a CDR2 with the amino acid sequence set forth in SEQ ID NO:68, and a CDR3 with the amino acid sequence set forth in SEQ ID NO:69; and the variable light chain of the anti-MuSK antibody comprises a CDR1 with the amino acid sequence set forth in SEQ ID NO:71, a CDR2 with the amino acid sequence set forth in SEQ ID NO:72, and a CDR3 with the amino acid sequence set forth in SEQ ID NO:73.

16. The anti-MuSK antibody of claim 7, wherein the variable heavy chain of the anti-MuSK antibody comprises a CDR1 with the amino acid sequence set forth in SEQ ID NO:75, a CDR2 with the amino acid sequence set forth in SEQ ID NO:76, and a CDR3 with the amino acid sequence set forth in SEQ ID NO:77; and the variable light chain of the anti-MuSK antibody comprises a CDR1 with the amino acid sequence set forth in SEQ ID NO:79, a CDR2 with the amino acid sequence set forth in SEQ ID NO:80, and a CDR3 with the amino acid sequence set forth in SEQ ID NO:81.

17. The anti-MuSK antibody of claim 7, wherein the variable heavy chain of the anti-MuSK antibody comprises a CDR1 with the amino acid sequence set forth in SEQ ID NO:83, a CDR2 with the amino acid sequence set forth in SEQ ID NO:84, and a CDR3 with the amino acid sequence set forth in SEQ ID NO:85; and the variable light chain of the anti-MuSK antibody comprises a CDR1 with the amino acid sequence set forth in SEQ ID NO:87, a CDR2 with the amino acid sequence set forth in SEQ ID NO:88, and a CDR3 with the amino acid sequence set forth in SEQ ID NO:89.

18. The anti-MuSK antibody of claim 7, wherein the variable heavy chain of the anti-MuSK antibody comprises a CDR1 with the amino acid sequence set forth in SEQ ID NO:91, a CDR2 with the amino acid sequence set forth in SEQ ID NO:92, and a CDR3 with the amino acid sequence set forth in SEQ ID NO:93; and the variable light chain of the anti-MuSK antibody comprises a CDR1 with the amino acid sequence set forth in SEQ ID NO:95, a CDR2 with the amino acid sequence set forth in SEQ ID NO:96, and a CDR3 with the amino acid sequence set forth in SEQ ID NO:97.

19. The anti-MuSK antibody of claim 7, wherein the antibody is a monoclonal antibody.

20. The anti-MuSK antibody of claim 7, wherein the antibody further comprises a radiolabel.
